(19) United States
(12) Patent Application Publication
(10) Pub. No.: US 2025/0205288 A1
(43) Pub. Date: Jun. 26, 2025

(54) DENTAL PULP AND DENTIN REGENERATION

(71) Applicant: University of Iowa Research Foundation, Iowa City, IA (US)

(72) Inventors: Dong Rim Seol, Iowa City, IA (US); Kyungsup Shin, Iowa City, IA (US); James A. Martin, Iowa City, IA (US); Venkat Ganesh, Iowa City, IA (US)

(21) Appl. No.: 18/990,921

(22) Filed: Dec. 20, 2024

Related U.S. Application Data
(60) Provisional application No. 63/614,149, filed on Dec. 22, 2023.

(51) Int. Cl. A61K 35/37; A61K 9/50

(52) U.S. Cl. CPC A61K 35/37; A61K 9/5068

(57) ABSTRACT

Provided herein are compositions and method to treat a subject for a dental pulp injury, disease or disorder or to regenerate dental pulp.

### PRIORITY

This application claims the benefit of the filing date of U.S. application No. 63/614,149, filed Dec. 22, 2023, the disclosure of which is incorporated by reference herein.

### GOVERNMENT SUPPORT

This invention was made with government support under DE030515 awarded by the National Institute of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Dec. 12, 2024, is named 875243US1.xml and is 118,327 bytes in size.

### BACKGROUND

Exosomes are vesicles of endocytic origin released by many cells. Exosomes are small vesicular structures averaging 40-120 nm (generally <250 nm) in diameter and are distinguished by their formation within cellular endosomal compartments known as multivesicular bodies (MVBs). Exosomes can contain proteins, peptides, and ribonucleic acids (RNAs).

### SUMMARY

Dental pulp is a highly vascularized soft tissue with a layer of odontoblasts at the inner dentin surface. One of the main physiological functions of dental pulp is to provide nutrition to dentin and detect unhealthy stimuli as a biosensor. Current endodontic therapy for dental caries, which is one of the most prevalent infectious diseases in the world [1], is a procedure for replacing vital pulp with synthetic materials via root canal therapy (RCT). Calcium hydroxide paste (CHP) and mineral trioxide aggregate (MTA) have been commonly used as pulp-capping materials due to their antibacterial properties, biocompatibility, and reparative dentin bridge formation [2-5]. However, CHP has several disadvantages for long-term use such as bacterial leakage into the dental pulp, poor cohesive strength, and high solubility [6]. Similarly, the drawbacks of MTA are its expensive cost, discoloration of the tooth, and long setting time [7]. Moreover, RCT-treated pulpless teeth can lose their ability to sense environmental changes and maintain dentin regeneration, ultimately compromising the mechanical integrity of the teeth. As an alternative, vital pulp therapy (VPT), which is defined as a restorative dental treatment that aims to preserve and maintain pulp tissue, is beneficial for young patients who have a high healing capacity for pulp regeneration [8,9]. Recently, the potential for successful VPT and pulp regeneration is increasing due to the use of mesenchymal stem cells (MSCs) that can differentiate into specialized cells [10-12]. However, the transplantation of MSCs incurs high costs and risks associated with ex vivo cell expansion [13].

Thus, there is an urgent need to develop alternative therapeutic methodologies for pulp regeneration.

Provided herein is a method to treat a subject for a dental pulp injury, disease or disorder comprising administering to a subject in need thereof a composition comprising exosomes obtained from dental pulp stem cells (DPSCs). Also provided herein is a method to regenerate dental pulp comprising administering to a subject in need thereof a composition comprising exosomes obtained from dental pulp stem cells (DPSCs). One aspect provides a method to treat a subject for a dental pulp injury, disease or disorder or to regenerate dental pulp comprising administering to a subject in need thereof a composition comprising engineered exosomes.

In some aspects, the exosomes are isolated from DPSCs cultured in growth media (DPSC-Exo-G), angiogenic differentiation media (DPSC-Exo-A), odontogenic differentiation media (DPSC-Exo-O) or a combination thereof. In some aspects, the exosomes display annexin A5 (ANXA5), tumor susceptibility gene 101 (TSG101), flotillin-1 (FLOT1), intercellular adhesion molecule 1 (ICAM), apoptosis-linked gene-2-interacting protein X (ALIX), epithelial cell adhesion molecule (EpCAM), CD63, CD81 or a combination thereof. In some aspects, the exosomes have a mean particle size about 80 nm to about 250 nm, including a mean particle size is about 104 nm or about 206 nm.

In some aspect, the exosomes comprise a nucleic acid sequence selected from the group consisting of a nucleic sequence of Table 1, Table 2, Table 3, Table 4, a nucleic acid sequence having at least about 90%6 sequence identity thereto and retaining activity associated with the microRNA (miRNA or miR) or a combination thereof. In some aspects the exosomes comprise a nucleic acid sequence selected from the group consisting of SEQ ID NOs: 1 to 9, a nucleic acid sequence having at least about 90% sequence identity thereto and retaining activity associated with the miRNA or a combination thereof. In some aspects, the composition comprises a nucleic acid sequence selected from the group consisting of SEQ ID NOs: 1 to 9 a nucleic acid sequence having at least about 90% sequence identity thereto and retaining activity associated with the miRNA or a combination thereof. In some aspects, the miRNA is present in a vector comprising a transcribable nucleic acid molecule encoding the miRNA operably linked to a promoter. In some aspects, the miRNA is independently present in the composition or contained with the exosome.

In some aspects, the composition further comprises hydrogel comprising fibrinogen and hyaluronic acid (HA). In some aspects, the exosomes are encapsulated in a hydrogel. In some aspects, the hydrogel comprises about 12.5% (w/v) fibrinogen and about 0.25% (w/v) hyaluronic acid (HA).

In some aspects, the subject is a mammal, such as a human.

In some aspects, the exosomes are isolated from mammalian cells, such as human.

### DRAWINGS

FIGS. 1A-1J. Characterization of rabbit dental pulp stem cells (DPSCs) and DPSC-derived exosomes (DPSC-Exos). (A, B) Tube formation as a marker of angiogenesis from DPSCs cultured in a regular medium (A) or angiogenic induction medium for 10 days (B). (C, D) Alizarin Red S staining as a marker of osteogenesis in osteogenic induction medium at 10 (C) and 20 (D) days. Exosomes were isolated from DPSCs cultured under a growth (Exo-G) or angiogenic differentiation condition (Exo-A). (E) Average size and concentration of Exo-G and Exo-A via Nanoparticle Tracking Analyzer (NTA). Red error bars indicate±standard error of the mean (n=3) (F) Exosome antibody array having eight positive markers (CD63, EpCAM: epithelial cell adhesion molecule, ANXA5: annexin A5, TSG101: tumor susceptibility gene 101, FLOT1: flotillin-1, ICAM: intercellular adhesion molecule 1, ALIX: programmed cell death 6 interacting protein, and CD81) and four controls including cis-Golgi matrix protein (GM130) as a negative marker for cellular contamination during exosome isolation, two positive controls (+Ctrl), and a blank control (Blank). (G, H) Scanning electron microscopy (SEM) images of Exo-G (G) and Exo-A (H). (I, J) Cellular uptake of Exo-G (I) and Exo-A (J) using PKH67 green fluorescence.

FIGS. 2A-2D. The effect of dental pulp stem cells-derived exosomes (DPSC-Exos) on cell toxicity, proliferation, and migration. Exosomes were isolated from DPSCs cultured under a growth (Exo-G) or angiogenic differentiation condition (Exo-A) and used at a concentration of 5×10⁷/mL (Exo-G-L or Exo-A-L), 5×10⁸/mL (Exo-G-M or Exo-A-M), or 5×10⁹/mL (Exo-G-H or Exo-A-H). (A) Cytotoxicity at 24 h (n=4-16). (B) Cell proliferation at 4 days (n=6). (C) Representative confocal images of migrated cells. FBS: fetal bovine serum. Green: Calcein AM. Scale bar: 200 μm. (D) Quantified fluorescence for cell migration (n=3).

FIG. 3. The effect dental pulp stem cells-derived exosomes (DPSC-Exos) on angiogenic differentiation. Exosomes (5×10⁸/mL) isolated from DPSCs cultured under a growth (Exo-G) or angiogenic differentiation condition (Exo-A) were treated in either basal or complete angiogenic induction medium (bAM or cAM) (n=4). Control: no exosome treatment. VEGFA: vascular endothelial growth factor A, FLT1: Fms-related tyrosine kinase 1 (known as VEGF receptor 1), and PECAM1: platelet and endothelial cell adhesion molecule 1.

FIGS. 4A-4E. MicroRNA (miRNA) profiling of rabbit (Oryctolagus cuniculus: ocu) dental pulp stem cells derived exosomes (DPSC-Exos) via next-generation sequencing (NGS). Exosomes were isolated from DPSCs cultured under growth (Exo-G) or angiogenic differentiation conditions (Exo-A). (A) Mature miRNAs with over 10,000 total read counts in Exo-G. (B) Mature miRNAs with over 10,000 total read counts in Exo-A. (C) Mature miRNAs with over 10,000 total read counts in both Exo-G and Exo-A. (D) Volcano plot of Exo-A versus Exo-G. Red: significantly up/down-expressed miRNAs. (E) The number of identified miRNAs.

FIGS. 5A-5C. Schematic of pulp regeneration in a rabbit partial pulpotomy model. A) Irreversible infected or inflamed pulpitis, B) Class V cavity with partial pulpotomy, C) Injection of DPSC-Exo with hydrogel. EN: enamel, D: dentin, P: pulp.

FIGS. 6A-6G. Characterization of DPSC-Exos. Nanoparticle tracking analysis of Exo-G (A), Exo-A (B), Exo-O (C), and summary of size and concentration (D). Exosome antibody array of Exo-G (E), Exo-A (F), and Exo-O (G).

FIGS. 7A-7C. Exosomal miRNA profile. PCA of mature miRNAs (A), Venn diagram of mature miRNAs (B), Summary of mature, hairpin, and novel miRNAs (C).

FIGS. 8A-8C. Effect of DPSC-Exos on cell toxicity (A), and proliferation (B), and migration (c). Exo-LL: 5×10⁶/ml, Exo-L: 5×10⁷/ml, Exo-M: 5×10⁸/ml, Exo-H: 5×10⁹/ml.

FIG. 9. Alkaline phosphatase (ALP) level in the conditioned media at 10 days.

### DESCRIPTION

Current endodontic therapy for dental caries, which are one of the most prevalent infectious diseases in the world, is a procedure for replacing the vital pulp with synthetic pulp-capping materials. Pulpless teeth can lose their functions to sense environmental changes and maintain dentin regeneration, and the synthetic materials have several disadvantages such as bacterial leakage into the dental pulp, poor cohesive strength, discoloration of tooth, and long setting time. As an alternative, vital pulp therapy (VPT), which is defined as a restorative dental treatment that aims to preserve and maintain pulp tissue, is beneficial for young patients who have high healing capacity for pulp regeneration (however, the current approach via pulp-capping materials is limited to tooth revitalization/revascularization with necrotic pulp). Potential for successful VPT and pulp regeneration is increasing due to the knowledge of mesenchymal stem cells (MSCs) that can differentiate into specialized cells. However, the transplantation of MSCs incurs high costs and risks associated with the ex vivo cell expansion. Consequently, a cell homing strategy which recruits endogenous dental pulp stem cells (DPSCs) is the effective approach in endodontics. Recently, exosomes have attracted attention due to their potential to promote intercellular communication leading to enhanced cell recruitment, differentiation to specific cell lineage, and tissue regeneration. In particular, conditioned medium or exosomes cultured under lineage-specific differentiation have use for angiogenesis and odontogenesis for pulp regeneration.

Provided herein is a pulp capping material system for vital pulp therapy of human dental pulp. Exosomes stimulate dental pulp regeneration by promoting DPSC chemotaxis and lineage-specific differentiation. In some embodiments, exosomes isolated from a subject, such as human or a rabbit DPSCs, are cultured under growth or lineage-specific differentiation conditions (odontogenesis or angiogenesis) and can be encapsulated in injectable hydrogel (which allows for addition of chemotactic factors like exosomes (and optionally a cell attractant), such as a fibrin/hyaluronic acid hydrogel which has temperature-sensitive gelation behavior at body temperature.

### Exosomes

### Isolation

Exosomes can be isolated as described herein. Further, the processes for identification, isolation, or characterization of an exosome are understood in the art (see e.g., Jensen 2010 RNA Exosome (Advances in Experimental Medicine and Biology Book 702), Springer, ISBN-10: 1441978402). Exosomes contain messenger RNA (mRNA) or microRNA (see e.g., Valadi et al. 2007 Nature Cell biology 9, 654-659).

An exosome described herein can be derived from dental tissue. In some embodiments, exosomes are derived from heterologous dental pulp (such as dental tissue that is heterologous to the subject (e.g., a patient) in which such exosomes will be used/administered for treatment). Exosomes are non-immunogenic, and therefore, heterologous exosomes are a viable source for treatment of patients.

Further, the stem cells, prior to exosome isolation, can be cultured under angiogenic or odontogenic differentiation conditions (as described herein or by protocols available to an art worker), following which exosomes can be isolated.

Additionally, exosomes, including engineered exosomes, can be generated by methods available to an art worker, such as by loading one or more miRNA(s) into exosomes including the steps of (1) incubation, (2) transfection, (3) physical (passive) treatments, and (4) in situ assembly and synthesis (see, for example, the review by Fu et al. Exosome engineering: Current progress in cargo loading and targeted delivery. NanoImpact 20 (2020) 100261, which is incorporated by reference herein).

Further methods of generating/making exosomes, including engineered exosomes, include manufacturing lipid nanoparticles (similar in structure to exosomes) loaded with synthesized miRs or other constituents that mimic the biologically active material in DPSC exosomes. These can be made in a laboratory setting without any need to harvest or culture cells (see, for example, Scheideler et al. Lipid nanocarriers for microRNA delivery. Chemistry and Physics of Lipids. 226 (2020) 104837, which is incorporated herein by reference).

Exosomes can have a mean particle size of about 80 to about 250 nm, including, including about 104 nm or about 206 nm.

An exosome described herein can be used in compositions or methods described herein alone, in combination with one or more other exosomes, miRNAs, RNAs, or polypeptides, as isolated or modified to contain less than an endogenous complement of miRNAs, RNAs, or polypeptides; or modified to contain more than an endogenous complement of miRNAs, RNAs, or polypeptides, including additional endogenous molecules or additional exogenous molecules.

microRNA (miRNA or miR)

Exosomes contain mRNA or microRNA.

As described herein, miRNA contained within a dental tissue-derived exosome can be used to treat a subject for a dental pulp injury, disease or disorder or to regenerate dental pulp. Exosome miRNAs can differ from miRNAs expressed by their parent cells.

A miRNA associated with exosomes from dental tissue can be used for a variety of effects associated with the exosome or for independent effects. A miRNA associated with exosomes from dental tissue can be used to treat a subject for a dental pulp injury, disease or disorder or to regenerate dental pulp.

Processes for identification and isolation of a miRNA are provided herein and are understood in the art (see e.g., Ochiya 2013 Circulating MicroRNAs: Methods and Protocols (Methods in Molecular Biology), Humana Press, ISBN-I 0: 1627034528). Exosome miRNA profiles can be determined according to conventional methods in the art.

A miRNA useful in a composition or method described herein can be identified or isolated from dental pulp stem cell exosomes (see e.g., Tables 1 to 4).

For example, a miRNA can include human sequences:

A miRNA can include SEQ ID NO: 1 (hsa-miR-21-5p): AGCUUAUCAGACUGAUGUUGA or a miRNA having at least about 80% (e.g., at least about 85%, 90%, 95%, or 99%) sequence identity thereto and retaining an activity associated with the miRNA.

A miRNA can include SEQ ID NO: 2 (hsa-miR-23b-3p): AUCACAUUGCCAGGGAUUACCAC or a miRNA having at least about 80% (e.g., at least about 85%, 90%, 95%, or 99%) sequence identity thereto and retaining an activity associated with the miRNA.

A miRNA can include SEQ ID NO: 3 (hsa-miR-24-3p): UGGCUCAGUUCAGCAGGAACAG or a miRNA having at least about 80% (e.g., at least about 85%, 90%, 95%, or 99%) sequence identity thereto and retaining an activity associated with the miRNA.

A miRNA can include SEQ ID NO: 4 (hsa-miR-122-5p): UGGAGUGUGACAAUGGUGUUUG or a miRNA having at least about 80% (e.g., at least about 85%, 90°/c, 95%, or 99%) sequence identity thereto and retaining an activity associated with the miRNA.

A miRNA can include SEQ ID NO: 5 (hsa-miR-143-3p: UGAGAUGAAGCACUGUAGCUC or a miRNA having at least about 80% (e.g., at least about 85%, 90%, 95%, or 99%) sequence identity thereto and retaining an activity associated with the miRNA.

A miRNA can include SEQ ID NO: 6 (hsa-miR-146a-5p): UGAGAACUGAAUUCCAUGGGUU or a miRNA having at least about 80% (e.g., at least about 85%, 90%, 95%, or 99%) sequence identity thereto and retaining an activity associated with the miRNA.

A miRNA can include SEQ ID NO: 7 (hsa-miR-199a-3p): ACAGUAGUCUGCACAUUGGUUA or a miRNA having at least about 80% (e.g., at least about 85%, 90%, 95%, or 99%) sequence identity thereto and retaining an activity associated with the miRNA.

A miRNA can include SEQ ID NO: 8 (hsa-miR-214-3p): ACAGCAGGCACAGACAGGCAGU or a miRNA having at least about 80% (e.g., at least about 85%, 90%, 95%, or 99%) sequence identity thereto and retaining an activity associated with the miRNA.

A miRNA can include SEQ ID NO: 9 (hsa-miR-221-3p): AGCUACAUUGUCUGCUGGGUUUC or a miRNA having at least about 80% (e.g., at least about 85%, 90%, 95%, or 99%) sequence identity thereto and retaining an activity associated with the miRNA.

A miRNA described herein can be used in a composition or method described herein alone, in combination with one or more other miRNA, RNA, or polypeptides, or in an exosome.

A miRNA described herein can be included in an expression vector, expression construct, plasmid, or recombinant nucleic acid construct. A vector, construct, or plasmid can include a transcribable nucleic acid molecule capable of being transcribed into a miRNA described herein. A transcribable nucleic acid molecule encoding a miRNA described herein can be operably linked to a promoter (e.g., an inducible promoter) functional in vitro or in vivo according to the species of the subject. A transcribable nucleic acid molecule encoding a miRNA described herein can be operably linked to a regulatory sequence.

A vector, construct, or plasmid encoding a miRNA described herein can be used to transform a host cell (e.g., in vitro transformation, ex vivo transformation, or in vivo transformation). A host cell transformed with a vector, construct, or plasmid encoding a miRNA described herein can be introduced (e.g., implanted) into a subject according to conventional techniques.

Compositions and methods described herein utilizing molecular biology protocols can be performed according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773: Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCR, ISBN-10: 3527310363; Baneyx (2004) Protein Expression Technologies, Taylor & Francis, ISBN-10: 0954523253).

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10: 0879695773; Green and Sambrook 2012 Molecular Cloning: A Laboratory Manual, 4th ed., Cold Spring Harbor Laboratory Press, ISBN-10: 1605500569; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

Constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of variant nucleotides or polypeptides having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. Conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16 6(log₁₀[Na⁺])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russell, 2006).

Exemplary nucleic acids which may be introduced to a host cell include, for example, DNA or RNA sequences or genes from another species, or even RNA, genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene, RNA or DNA is intended to refer to any gene, RNA or DNA segment that is introduced into a recipient cell, regardless of whether a similar gene, RNA or DNA may already be present in such a cell. The type of DNA/RNA included in the exogenous DNA/RNA can include DNA/RNA which is already present in the cell, DNA/RNA from another individual of the same type of organism, DNA/RNA from a different organism, or a DNA generated externally, such as a DNA/RNA sequence containing an antisense message of a gene, or a DNA/RNA sequence encoding a synthetic or modified version of a gene.

Methods of down-regulation or silencing genes are known in the art. For example, expressed protein activity can be down regulated or eliminated using antisense oligonucleotides, protein aptamers, nucleotide aptamers, and RNA interference (RNAi) (e.g., small interfering RNAs (siRNA), short hairpin RNA (shRNA), and microRNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulations

The agents, exosomes and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations can contain a therapeutically effective amount of a biologically active agent described herein, which can be in purified form, together with a suitable amount of carrier so as to provide the form for proper administration to the subject.

The formulation should suit the mode of administration. The agents of use with the current disclosure can be formulated by known methods for administration to a subject using several routes which include, but are not limited to, local/tooth cavity or defect. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

The agents, exosomes and compositions can be formulated with a hydrogel. Exosomes derived from dental pulp stem cells (DPSC-Exo) can be encapsulated in injectable fibrin/HA hydrogel which has temperature-sensitive gelation behavior at body temperature. Fibrin hydrogel supports cellular adhesion and chemotaxis, enabling uniform DPSCs to achieve even distribution in fibrin-filled defects (Rosso F, et al. J Cell Physiol 2005; 203(3):465-70; Swartz D D, et al. Am J Physiol Heart Circ Physiol 2005; 288(3):H1451-60). Moreover, it can affect the extent and stability of capillary tube formation (van Hinsbergh V W, et al. Ann N Y Acad Sci 2001; 936:426-37). A disadvantage of fibrin hydrogel is an increasing instability and solubility in long-term culture due to its fibrinolysis. Hyaluronic acid (HA), which is one of the components of the extracellular matrix (ECM) of connective tissue, can stabilize the fibrin hydrogel structure and inhibit fibrinolysis for DPSC therapies (Yu Y, et al. Arthritis Rheumatol 2015; 67(5):1274-85; Ahmadian E, et al. Int J Biol Macromol 2019; 140:245-54; Komorowicz E, et al. Matrix Biol 2017; 63:55-68). Thus, the hydrogel system can allow long-term release of chemotactic and differentiation factors from encapsulated DPSC-Exos.

As an example, a hydrogel comprises about 12.5% (w/v) fibrinogen (Tisseel Kit, Baxter), about 10 U/ml thrombin (Tisseel Kit, Baxter), and about 0.25% (w/v) hyaluronic acid (HA) (Gel-One®, Zimmer) (Yu Y, et al. Arthritis Rheumatol 2015; 67(5):1274-85). DPSC-Exos (such as about 1×10¹⁰) can be encapsulated in the hydrogel prior to use in methods described herein.

In some aspects, fibrin/hyaluronic acid (HA) hydrogel is used. The fibrin/HA can have two components: (A) about 25 mg/ml fibrinogen+exosomes, (B) about 5 mg/ml hyaluronic acid+thrombin. For example, 1:1 mixed, final concentrations of fibrinogen and hyaluronic acid can be 12.5 mg/ml and 2.5 mg/ml, respectively. In some embodiments, the hydrogel comprises 5-20 mg/ml fibrinogen and 1-5 mg/ml hyaluronic acid.

Further, the hydrogel system allows for the addition of chemotactic factors like exosomes that can improve DPSC recruitment. The temperature-sensitive hydrogel comprising fibrin/HA is an effective vehicle to enhance the retention of exosomes and the stability of microRNAs (miRNAs).

### Uses/Therapeutic Methods

Described herein are exosomes isolated from dental pulp stem cells (DPSCs) that can act as therapeutic agents. Exosomes provided herein can be used to treat a subject for a dental pulp injury, disease or disorder or regenerate dental pulp. Such diseases include, but are not limited to, pulpitis, pulp necrosis, and physical trauma to dental pulp (heat, mechanical, chemical (e.g., erosion). The patient/subject can be of any age, such as an infant, toddler, adolescent or adult.

Provided in the present disclosure is a process of treating dental pulp injury, disease or disorder in a subject in need administration of a therapeutically effective amount of composition described herein, so as to increase dental pulp in a target tooth (e.g., promote pulp regeneration).

A determination of the need for treatment will typically be assessed by a history and physical exam consistent with the structure or tooth defect at issue. Subjects with an identified need of therapy include those with a diagnosed dental pulp injury, disease or disorder. The subject is an animal, including, but not limited to, mammals, reptiles, and avians, including horses, cows, dogs, cats, sheep, pigs, and chickens, and human.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include but are not limited to exosomes or miRNA described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied electronically. Detailed instructions may not be physically associated with the kit; instead, a user may be directed to an Internet web site specified by the manufacturer or distributor of the kit.

### Definitions

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the example that follows represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example I

### Introduction

A cell homing strategy is an effective approach for pulp regeneration in endodontics, as it can recruit endogenous dental pulp stem cells (DPSCs) residing in the pulp tissue to the damaged site in need of repair [14]. The DPSCs have unlimited self-renewal with high clonogenicity and lineage-specific multipotent abilities [15,16]. These characteristics empower DPSCs to migrate locally to sites of injury where they proliferate and differentiate as needed to replace damaged tissue [17]. Unlike cell transplantation, this DPSC homing approach does not require multiple surgical procedures for cell harvesting and transplantation.

Exosomes have attracted attention due to their potential to promote intercellular communication leading to enhanced cell recruitment, differentiation to specific cell lineage, and tissue repair [18-22]. Exosomes represent a mode of intercellular communication as they contain a variety of bioactive molecules including deoxyribonucleic acid (DNA), ribonucleic acid (RNA), lipids, and proteins [23]. In particular, microRNAs (miRNAs) have been increasingly recognized for their therapeutic potential [24]. Recent studies have demonstrated that damaged tissues are repaired by the paracrine signaling of exosomes rather than direct proliferation and differentiation [25]. This paracrine effect implies that exosome therapy has a clinical advantage over stem cell transplantation in terms of immune response and tumorigenesis. In addition, MSCs are promising sources of exosomes. Recently, exosomes isolated from MSCs have shown therapeutic potential in heart, skin, and hyaline cartilage regeneration [18, 21, 22, 26, 27]. A series of prior studies have shown DPSC-derived exosome (DPSC-Exo) enhanced cell recruitment and angiogenesis [28-30].

Provided herein are exosomes isolated from undifferentiated or angiogenic differentiated DPSCs that play a role in inducing cell migration and angiogenesis of naïve DPSCs, respectively, and play a role in in vitro cell homing and angiogenesis for pulp regeneration.

### Materials and Methods

### Isolation of DPSCs

Two batches of the incisor pulp tissues obtained from New Zealand White rabbit cadavers (approximately 10 months old) were harvested to isolate DPSCs. Under sterile conditions, the dental pulp was minced into approximately 1 mm³ and plated in a culture dish. The tissue fragments and migrated cells were cultured in alpha minimum essential medium (α-MEM; Thermo Fisher Scientific, Waltham, MA, USA) supplemented with 10% fetal bovine serum (FBS; Thermo Fisher Scientific), 50 μg/mL L-ascorbic acid 2-phosphate trisodium salt (FUJIFILM Wako Chemicals, Richmond, VA, USA), 100 U/mL penicillin-streptomycin (Thermo Fisher Scientific), and 2.5 μg/mL amphotericin B (Sigma-Aldrich, St. Louis, MO, USA) in a hypoxic culture condition (5% O₂/CO₂ at 37° C.) [66]. Additionally, the cells were cultured in angiogenic differentiation induction media, EGM™-2 endothelial cell growth medium (Lonza, Bend, OR USA).

### Characterization of DPSCs

Characterization of DPSCs (3rd-5th passage) was examined by multipotential differentiation. Angiogenic differentiation was evaluated using an endothelial tube formation assay (Cell Biolabs, San Diego, CA, USA) according to the manufacturer's instructions. In brief, 1.5×10⁴ cells cultured in angiogenic induction medium for 10 days were seeded in a 16-well chamber slide (Thermo Fisher Scientific) coated with 50 μL extracellular matrix (ECM) gel. After 4 h, the cells were incubated with 1× Staining solution and imaged by an Olympus FV1000 confocal microscope (Olympus, Center Valley, PA, USA). For odontogenesis, 1.5×10⁴ cells were cultured with osteogenic differentiation medium (ScienCell, Carlsbad, CA, USA) in a 6-well plate, and Alizarin Red S staining (Sigma-Aldrich) was used to evaluate the cellular calcium deposit at 10 and 20 days. The cells fixed in 4% formaldehyde were stained with 40 mM Alizarin Red S for 30 min (min) and imaged by an Olympus BX60 microscope (Olympus).

### Isolation of DPSC-Exos

Exosomes were isolated from rabbit DPSCs cultured under a growth (Exo-G) or angiogenic differentiation (Exo-A) condition. For Exo-A isolation, the cells were cultured in an angiogenic differentiation medium for 10 days. The induction medium was replaced with α-MEM growth medium containing 10% exosome-depleted FBS, and the conditioned medium (CM) was collected after 48 h. CM collected from non-inducted DPSCs was prepared for Exo-G isolation. DPSC-Exos were isolated by a precipitation method using ExoQuick-TC™ (System Biosciences, Palo Alto, CA, USA). In brief, CM was centrifuged at 3000×g for 15 min to remove cells and cell debris. The supernatant was mixed with an ExoQuick-TC™ solution at 4° C. overnight. After centrifugation, the exosome pellet was resuspended in PBS and stored at −80° C. before use.

### Nanoparticle Tracking Analyzer (NTA)

NTA was utilized to obtain the size distribution and concentration of DPSC-Exos using a NanoSight instrument (Malvern Panalytical, Westborough, MA, USA). Exosomes suspended in PBS were placed into a sonic bath at 30° C. for 10 min. The samples were then diluted for analysis with 0.1 μm-filtered PBS and injected into the laser viewing module via an automated syringe pump. The sample was illuminated using project-specific optical configuration settings according to the manufacturer's protocol. Nanoparticles were tracked individually, counted, and sized via a translational diffusion coefficient. Triple samples were analyzed with the following parameters: syringe flow rate=13, detection threshold=3, track length=10, and camera=9-15. The data are reported in a number weighted distribution. Latex beads of 100 nm diameter were used as a reference.

### Exosome Antibody Array

The identification of DPSC-Exos was validated using an Exo-Check™ exosome antibody array (System Biosciences). The array had eight positive markers (CD63, EpCAM: epithelial cell adhesion molecule, ANXA5: annexin A5, TSG101: tumor susceptibility gene 101, FLOT1: flotillin-1, ICAM: intercellular adhesion molecule 1, ALIX: programmed cell death 6 interacting protein, and CD81) and four controls including cis-golgi matrix protein (GM130) as a negative marker for cellular contamination during exosome isolation, two positive controls (+ctrl), and a blank control. According to the manufacturer's protocol, 50 μg DPSC-Exos suspended in PBS were mixed with 10% (v/v) lysis buffer and 1 μL labeling reagent. After 30 min incubation at room temperature (RT), the excess labeling reagent was removed using a column filter. A membrane was immersed in the mixture of labeled exosome lysate and 5 mL blocking buffer at 4° C. overnight on a shaker. After washing, the membrane was developed with WesternBright Sirius HRP substrate (Advansta, Menlo Park, CA, USA).

### Scanning Electron Microscopy (SEM)

DPSC-Exos were fixed in 2.5% glutaraldehyde in 0.1 M sodium cacodylate and loaded on a clean silicon wafer (Ted Pella, Redding, CA, USA) on the top of a 37° C. hotplate. The wafer was covered with 100% hexamethyldisilazane (Ted Pella) for 10 min and then washed with PBS. After drying overnight, the samples were mounted on an aluminum stub (Ted Pella) with double-sided carbon tape and processed for an iridium sputter coating (Q150R-S, Quorum Technologies, Laughton, UK) to make the surface conductive. The final product was imaged in a high-resolution SEM (S-4800, Hitachi High-Tech, Ibaraki, Japan).

### Exosome Labeling and Cellular Uptake

DPSC-Exos were labeled with a PKH67 green lipid membrane dye (Sigma-Aldrich) according to the manufacturer's instructions. Briefly, 5×10⁸/mL exosomes suspended in 1 mL diluent C were mixed with 4 μL PKH67 and incubated for 5 min at room temperature. An equal volume of 5% bovine serum albumin was added to stop the labeling, and then the mixture was centrifuged with 110,000 g for 2 h at 4° C. After washing with PBS and repeating centrifugation, the labeled exosomes were co-cultured with DPSCs for 48 h and imaged by an Olympus FV1000 confocal microscope (Olympus, Center Valley, PA, USA). Exosomes without PKH67 labeling were used for a negative control.

### Cell Toxicity

DPSCs were seeded at a density of 1×10⁴ (200 μL) in 96-well plates. The next day, the cells were treated with various concentrations of DPSC-Exos (Exo-L: 5×10⁷/mL, Exo-M: 5×10⁸/mL, or Exo-H: 5×10⁹/mL) in a serum-free culture medium. Vehicle controls, 4.3%, 9.0%, and 16.3% (v/v) PBS, and no exosome treatment control, were prepared because the final products of exosomes were diluted in PBS. After 24 h, cytotoxicity was determined by CellTiter 96® Aqueous One Solution (Promega, Madison, WI, USA). One Solution Reagent 20 μL) was added into each well of the 96-well plates (100 μL medium), and the plates were incubated at 37° C. in a humidified incubator for 4 h. The absorbance was recorded at 490 nm using a 96-well plate reader (Infinite® 200 PRO, TECAN, Mannedorf, Switzerland).

### Cell Proliferation

The cells were seeded at a density of 1×10⁴ (200 μL) in 48-well plates. After 2 h for cell adhesion, the media were replaced with 200 μL of treatment groups: controls (serum-free medium or serum-free medium with 10% exosomes-depleted FBS) and 5×10⁷/mL or 5×10⁸/mL DPSC-Exos (Exo-G or Exo-A). The media were changed every two days. At day 4, cell viability was evaluated using CellTiter 96® Aqueous One Solution (Promega).

### Cell Migration

The cells (1×10⁴/100 μL) were added to the polycarbonate membrane inserts of 24-well Transwell® plates with an 8 μm pore (Corning, Corning, NY, USA). In the reservoirs, 600 μL of serum-free medium or DPSC-Exos (5×10⁷/mL and 5×10⁸/mL Exo-G or Exo-A) were added. Following 48 h incubation, the inserts were swabbed with sterile cotton tips to remove non-migrated cells and washed twice in Hanks' Balanced Salt Solution (HBSS). The inserts were stained with Calcein AM (1:1000 dilution; Thermo Fisher Scientific) and imaged by a confocal microscope. Separately, the migrated cells were digested in papain digestion buffer (1 mg/mL papain, 5 mM L-cysteine hydrochloride acid, 100 mM disodium hydrogen phosphate, 5 mM ethylenediaminetetraacetic acid salt) for 2 h and quantified using a Quant-iT™ PicoGreen® dsDNA Assay kit (Thermo Fisher Scientific). Fluorescence was measured on the plate reader at 480 nm excitation and 520 nm emission.

### Angiogenic Differentiation

A total of six groups were prepared to evaluate the effect of DPSC-Exos on angiogenic differentiation: (1) basal angiogenic (bAM; EGMTM-2 endothelial cell growth medium (Lonza, Bend, OR, USA)) differentiation medium without growth factors, (2) bAM+Exo-G, (3) bAM+Exo-A, (4) completed angiogenic (cAM; bAM+growth factors; growth factors: Hydrocortisone, hFGF-B (human fibroblastic growth factor beta), VEGF (vascular endothelial growth factor), IGF-1 (insulin-like growth factor 1), hEGF (human epidermal growth factor), and heparin) differentiation medium with growth factors, (5) cAM+Exo-G, and (6) cAM+Exo-A. DPSCs were seeded at a density of 3×10⁵ (2 mL) in 6-well plates, and the medium with or without 5×10⁸ DPSC-Exos was replaced every 2 days. After 7 days, the cells were trypsinized for gene expression analysis.

### Real-Time Polymerase Chain Reaction (RT-PCR)

The cells were lysed to collect ribonucleic acid (RNA) using an RNAqueous™ Total RNA Isolation kit (Thermo Fisher Scientific) for reverse transcription. A qualitative and quantitative assessment of RNA samples was performed with a Nanodrop™ 2000 spectrophotometer (Thermo Fisher Scientific) prior to complementary deoxyribonucleic acid (cDNA) synthesis using a SuperScript™ VILO™ cDNA Synthesis kit (Thermo Fisher Scientific). The Taqman™ gene expression assay was performed using a QuantStudio™ 3 RT-PCR System (Applied Biosystems, Foster City, CA, USA) in a 10 μL PCR reaction mix containing 2 μL of cDNA template and an appropriate concentration of the Taqman™ Advanced Master Mix and probes as recommended by the manufacturer. We used species specific, premade probes (Thermo Fisher Scientific) labeled with a fluorescein (FAM) reporter and a minor groove binder (MGB) quencher both for housekeeping (GAPDH: Oc03823402_g1) and three target genes, vascular endothelial growth factor A (VEGFA: Oc03395999_m1), Fms-related tyrosine kinase 1 (FLT1: Oc06783656_s1), and platelet and endothelial cell adhesion molecule 1 (PECAM1: Oc06726473_ml). The relative changes in gene expression levels were calculated by the comparative CT (DDCT) method [67].

### Next-Generation Sequencing (NGS)

Two batches of rabbit DPSC-Exos were prepared for NGS analysis. Exosomal RNAs were extracted and quantified by a Bioanalyzer Small RNA Assay kit (Agilent, Santa Clara, CA, USA). NGS libraries were prepared and sequenced on a HiSeq® Sequencing System (Illumina, San Diego, CA, USA) with 150 bp paired end reads at an approximate depth of 10-15 million reads per sample in System Biosciences. Raw data were analyzed in the Bioinformatics Division of the Iowa Institute of Human Genetics (IIHG). To quantitate known small RNAs and to identify novel miRNAs, we used the Nextflow-based workflow nf-core/smmaseq (version v1.1.0, https://github.com/nf-core/smrnaseq) (Accessed on 27 Apr. 2022) as a small-RNA sequencing analysis pipeline. Rabbit (Oryctolagus cuniculus, build oryCun2) genome sequence (fasta) and annotation (gff) were downloaded from Ensembl (http://ftp.ensembl.org/pub/release-100) (Accessed on 27 Apr. 2022). Sequences for mature and hairpin small RNAs were downloaded from miRBase (https://www.mirbase.org/ftp/CURRENT/) (Accessed on 27 Apr. 2022). The adaptor protocol used was ‘qiaseq’, but for all other parameters, the default values were used. In brief, the workflow uses FastQC for assessing read quality, Trim Galore! for adapter trimming, seqcluster for read collapsing, and Bowtie1 for aligning reads to small RNA sequences. Read counts were imported into R and normalized using DESeq2. To determine differential expression, a model incorporating all the experimental factors was created, and Wald tests were used to compute statistical metrics. A small RNA was considered to have a statistically significant change in expression if the False Discovery Rate (FDR) was less than 10%. Results from the differential expression analysis were visualized using a volcano plot created with the ggplot2 package in R. To identify novel miRNAs, sequence read files (fastq) from samples in the same group were first concatenated together and then ran through the same nfcore/smrnaseq workflow as above with the miRDeep2 software used for novel miRNA identification. The results from miRDeep2 were filtered to include only potential novel miRNAs with miRDeep2 scores greater than or equal to 4 (higher quality).

### Statistics

The scatter plots were expressed as the mean values with the standard deviation using GraphPad Prism (Version 8.1.2; San Diego, CA, USA). Data were compared by one-way ANOVA with the Tukey post-hoc test using SPSS Statistics software (Version 28; IBM, Armonk, NY, USA). Statistical significance was set at p<0.05.

### Results

### Characterization of DPSCs and DPSC-Exos

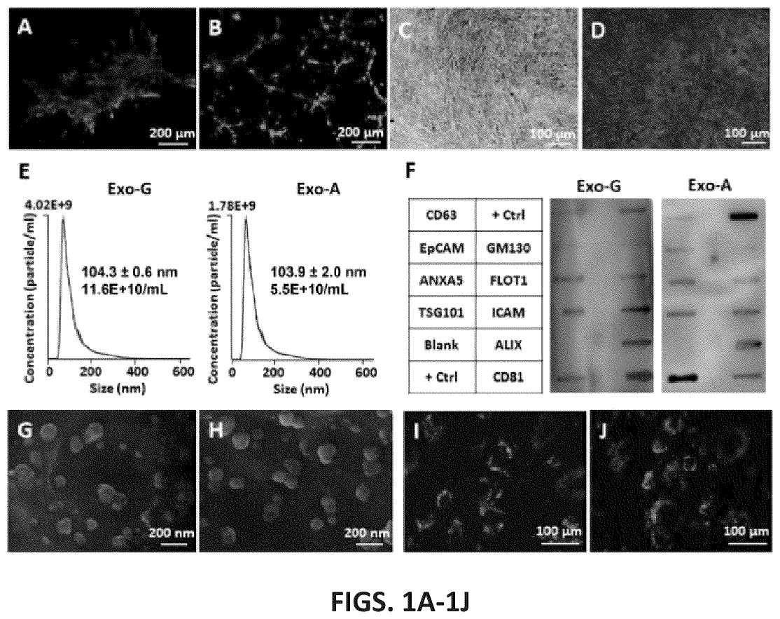

The stem cell characterization of rabbit DPSCs was validated by angiogenic and odontogenic multipotential differentiation. In an endothelial tube formation assay, elongated DPSCs formed capillary-like networks when they were pre-cultured in an angiogenic induction medium (FIG. 1B), whereas non-induced cells were clustered together without any tube formation (FIG. 1A). In odontogenic differentiation, strong deposits of calcium were observed time-dependently in Alizarin Red S staining (FIG. 1C, D).

Exosomes were isolated from DPSCs cultured under a growth (Exo-G) or angiogenic differentiation (Exo-A) condition. The characterization of exosomes was performed by a Nanoparticle Tracking Analyzer (NTA), Exo-Check™ exosome antibody array, scanning electron microscope (SEM), and cellular uptake assay. The concentration of Exo-G (11.6×10¹⁰ particles/mL) was approximately two times higher than that of Exo-A (5.5×10¹⁰ particles/mL), while the mean size of particles was similar (104.3 nm Exo-G and 103.9 nm Exo-A) (FIG. 1E). Both Exo-G and Exo-A showed a positive expression of ANXA5, TSG101, FLOT1, ICAM, ALIX, and CD81 as exosome markers, and a negative expression of GM130 as a negative marker (FIG. 1F). Under SEM, both exosomes had a spheroid shape approximately 100 nm in size (FIG. 1G, H). In an uptake assay, both types of exosomes labeled with PKH67 green fluorescence highly internalized into the DPSCs (FIG. 11, J).

### Effect of DPSC-Eros on Cell Toxicity, Proliferation, Migration, and Angiogenic Differentiation

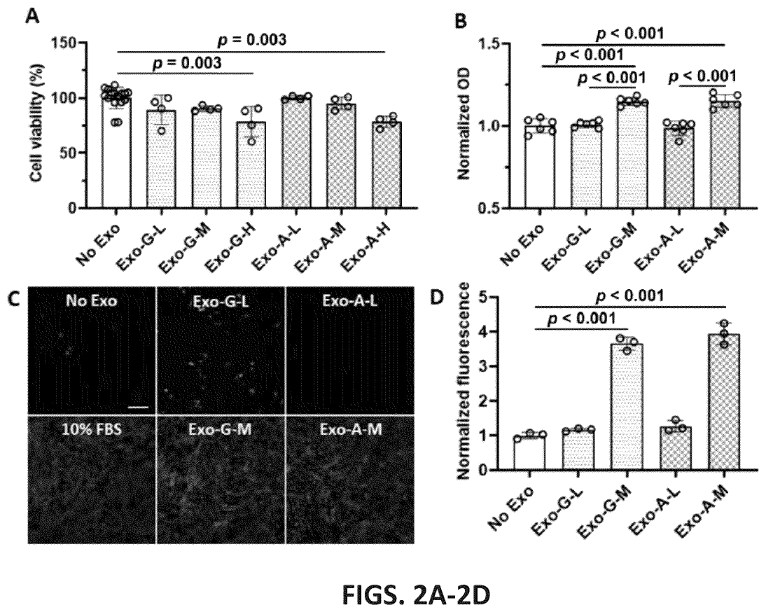

In the cytotoxicity test, vehicle controls (4.3%, 9.0%, and 16.3% (v/v) phosphate buffered saline (PBS)) did not impact cell viability. Therefore, all data from vehicle controls and no exosome treatment control were pooled together. There was no significant cell death in less than 5×10⁸ exosomes. However, the viability was significantly decreased in both 5×10⁹ Exo-G (p=0.003) and Exo-A (p=0.003) (FIG. 2A). For 4 days, 5×10⁸ exosome treatments resulted in a slight increase in cell proliferation in both Exo-G (p<0.001) and Exo-A (p<0.001) (FIG. 2B). In a migration assay using Transwell® plates, exosome treatment with a concentration of 5×10⁸ exosomes promoted dramatic cell migration in both groups of Exo-G (p<0.001) and Exo-A (p<0.001) (FIG. 2C, D). The effect was similar for a positive control which contained 10% (v/v) exosome depleted FBS. In contrast, a low concentration of exosomes (5×10⁷) did not affect cell migration.

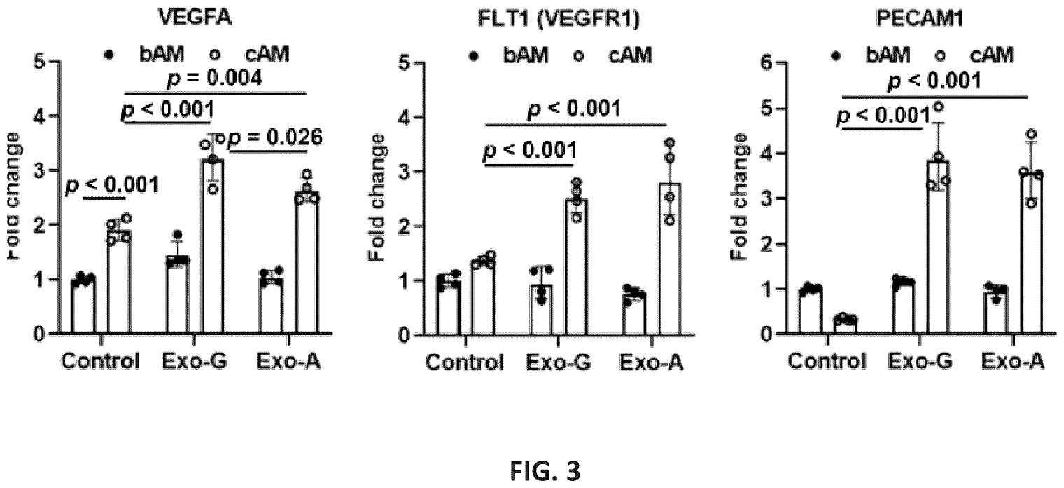

To evaluate the effect of DPSC-Exos on angiogenic differentiation, DPSCs were treated with 5×10⁸/mL exosomes in basal or complete induction media (bAM or cAM, respectively) for 7 days. There was no effect of DPSC-Exos on the expression of angiogenic markers when cultured in bAM (FIG. 3). On the other hand, angiogenic markers of VEGFA, FLT1 (VEGFR1), and PECAM1 were highly expressed when treated with Exo-G and Exo-A in cAM. Both exosome groups showed statistically higher expressions of VEGFA (Exo-G versus control: p<0.001 and Exo-A versus control: p=0.004), FLT1 (Exo-G versus control: p<0.001 and Exo-A versus control: p<0.001), and PECAM1 (Exo-G versus control: p<0.001 and Exo-A versus control: p<0.001). The degree of fold changes between Exo-G and Exo-A was similar in FLT1 and PECAM1, while Exo-G induced a higher expression of VEGFA (p=0.026 versus Exo-A).

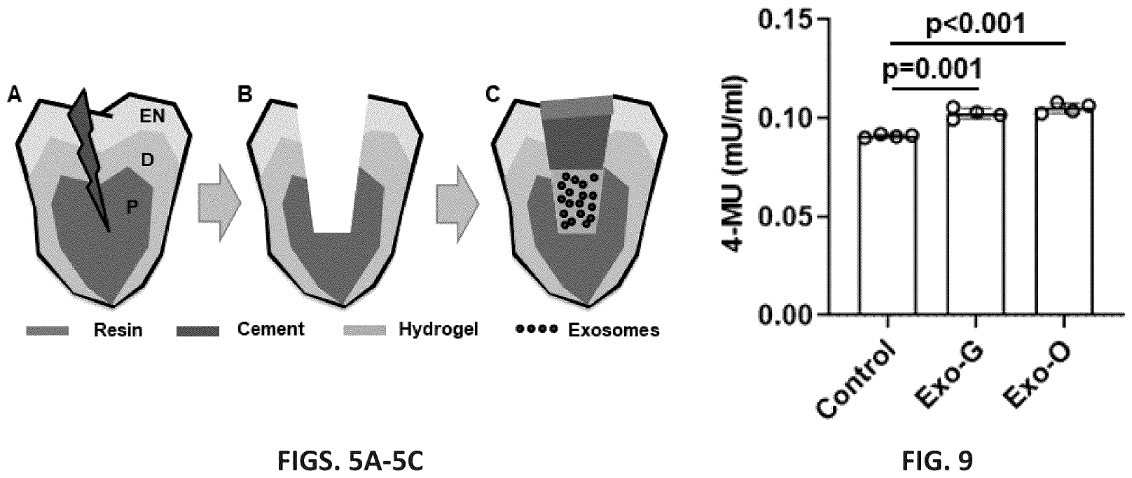

For in-vivo efficacy of DPSC-Exos, a partial pulpotomy animal model, which can mimic clinical conditions in human (FIG. 5), can be used. For example, about 1×10¹⁰ DPSC-Exo-G, -A, -O or a combination thereof can be encapsulated in hydrogel comprising about 12.5% (w/v) fibrinogen (Tisseel Kit, Baxter) and about 0.2% (w/v) HA (Gel-One®, Zimmer). One ml hydrogel can be incubated in 20 ml phosphate-buffered saline (PBS) at 37° C. with 50 rpm shaking. The supernatant (1 ml) can be collected at 0.25, 1, 3, 5, and 10 days, and the numbers of exosomes can be measured by nCS1.

Exosomal miRNA Profile

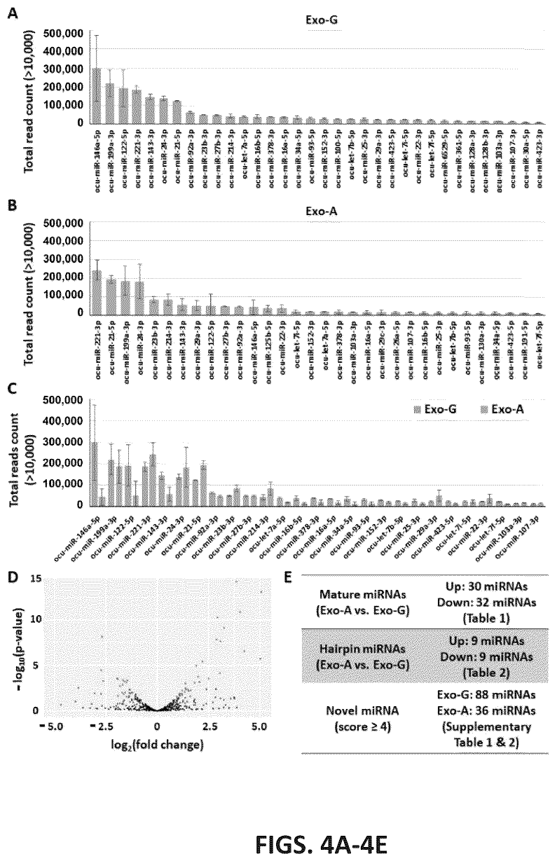

In NGS analysis, a total of 474 mature and 254 hairpin miRNAs were found in Exo-G and a total of 459 mature and 253 hairpin miRNAs were found in Exo-A. FIG. 4A, B show highly expressed miRNAs with over 10,000 total read counts in Exo-G and Exo-A, respectively. Mature miRNAs with over 10,000 total read counts in both Exo-G and Exo-A were shown in FIG. 4C. In particular, four miRNAs including ocu-miR-199a-3p, ocu-miR-221-3p, ocu-miR-24-3p, and ocu-miR-21-5p were detected in over 100,000 total read counts. In contrast, there was a distinct expression of miRNA levels in Exo-A when compared with that in Exo-G (FIG. 4D). Exo-A isolated under angiogenic differentiation conditions included 30 up-regulated (ocu-miR-708-5p, ocu-miR-205-5p, ocu-miR-708-3p, etc.) and 32 down-regulated (ocu-miR-146a-5p, ocu-miR-503-5p, ocu-miR-20b-5p, etc.) mature miRNAs (FIG. 4E). There were 18 hairpin miRNAs that significantly changed in Exo-A, of which 9 hairpin miRNAs (ocu-miR-708-5p, ocu-miR-205-5p, ocu-miR-708-3p, etc.) increased and 9 hairpin miRNAs (ocu-miR-708-5p, ocu-miR-205-5p, ocu-miR-708-3p, etc.) decreased. All differentially expressed mature and hairpin miRNAs are listed in Tables 1 and 2, respectively. Exosomal miRNAs with a significant expression of both mature and hairpin miRNAs in Exo-A were ocu-miR-708 (up), ocu-miR-134 (up), ocu-miR-125b (up), ocu-miR-140 (up), ocu-miR-29b (up), ocu-miR-214 (up), ocu-miR-574 (down), ocu-miR-503 (down), ocu-miR-30a (down), ocu-miR-146a (down), and ocu-miR-671 (down). In addition, 88 and 36 novel miRNAs were identified in Exo-G and Exo-A, respectively (Tables S1 and S2).

TABLE 1
List of significantly up / down-expressed mature
microRNAs (miRNAs): Exo-A versus Exo-G.

| | log2(Fold | | | log2(Fold | |
|---|---|---|---|---|---|
| miRNA | Change) | p-Value | miRNA | Change) | p-Value |
| ocu-miR-708-5p | 5.06 | 0.0000 | ocu-miR-146a-5p | −273 | 0.0000 |
| ocu-miR-205-5p | 4.24 | 0.0000 | ocu-miR-503-5p | −269 | 0.0000 |
| ocu-miR-708-3p | 4.03 | 0.0000 | ocu-miR-20b-5p | −263 | 0.0005 |
| ocu-miR-885-5p | 3.85 | 0.0000 | ocu-miR-18a-3p | −1.97 | 0.0003 |
| ocu-miR-24-2-5p | 3.24 | 0.0000 | oro-miR-18a-5p | −1.92 | 0.0003 |
| ocu-miR-29c-3p | 3.07 | 0.0000 | ocu-miR-122-5p | −1.91 | 0.0223 |
| ocu-let-7i-3p | 3.04 | 0.0001 | ocu-miR-421-3p | −1.81 | 0.0019 |
| ocu-miR-574-3p | 2.92 | 0.0000 | ocu-miR-350-5p | −1.72 | 0.0099 |
| ocu-miR-874-3p | 2.88 | 0.0000 | ocu-miR-1307-3p | −1.69 | 0.0013 |
| ocu-miR-134-5p | 2.50 | 0.0003 | ocu-miR-98-5p | −1.68 | 0.0004 |
| ocu-miR-U5b-Sp | 2.20 | 0.0000 | ocu-miR-432-5p | −1.64 | 0.0147 |
| ocu-miR-34c-3p | 2.06 | 0.0006 | ocu-miR-100-5p | −1.60 | 0.0005 |
| ocu-miR-140-3p | 1.99 | 0.0002 | ocu-miR-34a-5p | −1.58 | 0.0094 |
| ocu-miR-342-3p | 1.90 | 0.0000 | ocu-miR-16b-5p | −1.51 | 0.0008 |
| ocu-miR-145-5p | 1.82 | 0.0008 | ocu-miR-20a-5p | −1.50 | 0.0271 |
| ocu-miR-150-5p | 1.63 | 0.0053 | ocu-miR-30a-5p | −1.46 | 0.0004 |
| ocu-miR-148a-5p | 1.61 | 0.0251 | ocu-miR-93-5p | −1.38 | 0.0238 |
| ocu-miR-532-3p | 1.52 | 0.0065 | ocu-miR-6529-5p | −1.37 | 0.0045 |
| ocu-miR-30b-5p | 1.50 | 0.0046 | ocu-miR-143-3p | −1.37 | 0.0104 |
| ocu-miR-31-5p | 1.40 | 0.0044 | ocu-miR-652-3p | −1.36 | 0.0241 |
| ocu-miR-214-5p | 1.37 | 0.0031 | ocu-miR-574-5p | −1.33 | 0.0123 |
| ocu-miR-222-3p | 1.37 | 0.0010 | ocu-miR-7a-5p | −1.32 | 0.0047 |
| ocu-miR-323a-3p | 1.34 | 0.0082 | ocu-miR-151-3p | −1.26 | 0.0102 |
| ocu-miR-502a-3p | 1.21 | 0.0126 | ocu-miR-15b-3p | −1.23 | 0.0097 |
| ocu-miR-130a-3p | 1.21 | 0.0185 | ocu-miR-671-5p | −1.21 | 0.0101 |
| ocu-miR-12092-3p | 1.20 | 0.0055 | ocu-let-7f-5p | −1.09 | 0.0268 |
| ocu-miR-29b-3p | 1.13 | 0.0128 | ocu-let-7a-5p | −1.08 | 0.0220 |
| ocu-miR-29a-3p | 1.10 | 0.0216 | ocu-miR-423-5p | −1.06 | 0.0259 |
| ocu-miR-335-5p | 1.07 | 0.0174 | ocu-miR-125b-3p | −1.06 | 0.0146 |
| ocu-miR-181b-5p | 1.07 | 0.0143 | ocu-miR-361-5p | −1.04 | 0.0240 |
| | | | ocu-miR-128a-3p | −1.04 | 0.0161 |
| | | | ocu-miR-128b-3p | −1.04 | 0.0161 |

TABLE 2
List of significantly up/down-expressed hairpin
microRNAs (miRNAs): Exo-A versus Exo-G.

| | log2(Fold | | | log2(Fold | |
|---|---|---|---|---|---|
| miRNA | Change) | p-Value | miRNA | Change) | p-Value |
| ocu-mir-708* | 6.70 | 0.0000 | ocu-mir-574* | −3.05 | 0.0007 |
| ocu-mir-134* | 3.06 | 0.0001 | ocu-mir-503* | −2.68 | 0.0000 |
| ocu-mir-125b-2* | 2.65 | 0.0000 | ocu-mir-590 | −2.31 | 0.0133 |
| ocu-mir-140* | 2.24 | 0.0000 | ocu-mir-30a* | −1.94 | 0.0001 |
| ocu-mir-29b-2* | 2.04 | 0.0006 | ocu-mir-146a* | −1.93 | 0.0041 |
| ocu-mir-29b-1* | 1.95 | 0.0011 | ocu-mir-671* | −1.77 | 0.0014 |
| ocu-mir- 214* | 1.54 | 0.0003 | ocu-mir-181b-2 | −1.49 | 0.0112 |
| ocu-mir- 26a | 1.35 | 0.0013 | ocu-mir-152 | −1.38 | 0.0106 |
| ocu-mir- 23b | 1.05 | 0.0132 | ocu-mir-155 | −1.13 | 0.0104 |

*Significant expression of both mature and hairpin miRNAs.

TABLE 3
List of top 10 mature and hairpin microRNAs (miRNAs) in Exo-G.

| | Total read | | Total read |
|---|---|---|---|
| Mature miRNAs | counts | Hairpin miRNAs | counts |
| ocu-miR-146a-5p | 296,149.6 | ocu-miR-214 | 20,367.4 |
| ocu-miR-199a-3p | 217,998.9 | ocu-miR-503 | 10,826.0 |
| ocu-miR-122-5p | 190,513.3 | ocu-miR-199a-1 | 5,689.2 |
| ocu-miR-221-3p | 185,003.3 | ocu-miR-199a-2 | 5,687.8 |
| ocu-miR-143-3p | 143,493.5 | ocu-miR-23b | 5,641.1 |
| ocu-miR-24-3p | 137,071.1 | ocu-let-7b | 5,052.3 |
| ocu-miR-21-5p | 122,765.1 | ocu-miR-142 | 3,211.0 |
| ocu-miR-92a-3p | 61,986.4 | ocu-miR-34a | 2,377.4 |
| ocu-miR-23b-3p | 49,175.0 | ocu-miR-146a | 1,816.3 |
| ocu-miR-27b-3p | 47,342.7 | ocu-let-7i | 1,375.5 |

SEQ ID NOS: 10-97 are present in Table S1 below.

SUPPLEMENTARY TABLE S1
List of novel miRNAs in Exo-G.

| | | Total | | | | |
|---|---|---|---|---|---|---|
| miRDeep | Consensus Mature | read | | | | |
| 2 score | sequence | counts | Chromosome | Start | Stop | Strand |
| 3379.5 | GCTGTGATGGCCGAGTGG | 6656 | 11 | 30795998 | 30796065 | + |
| 1604.3 | GAAGTCGCAGCTCTCTGGGC | 3143 | 14 | 163848014 | 163848071 | − |
| 1017.7 | GGGGATGTAGCTCAGTGG | 2002 | 11 | 31944616 | 31944678 | + |
| 694.5 | TCAAAAAGTCGGCGGGAAATGC | 1359 | GL018776 | 1023377 | 1023447 | − |
| 590.5 | AGCCAGCTCTGTGGCGCAGTGG | 1164 | 7 | 34972538 | 34972589 | + |
| 350 | TTCCTGTGCTGTTCTCGGTAG | 684 | GL018700 | 9256430 | 9256486 | − |
| 350 | TTCCTGTGCTGTTCTCGGTAG | 684 | GL018700 | 9286959 | 9287015 | − |
| 344 | TTCCTGTGCTGTTCTCGGTAG | 672 | GL018700 | 9253158 | 9253214 | − |
| 343.9 | TTCCTGTGCTGTTCTCGGTAG | 672 | GL018700 | 9246701 | 9246757 | − |
| 343.9 | TTCCTGTGCTGTTCTCGGTAG | 672 | GL018700 | 9272391 | 9272447 | − |
| 242.1 | TGGGAACCTTGGGCAGGAAGT | 471 | GL018789 | 1006726 | 1006784 | − |
| 198.6 | GTGACCTGTGGTGCTGGTGG | 394 | GL018724 | 1521599 | 1521649 | − |
| 196.9 | TACTCAGAGCGGGACTGGAATGT | 383 | GL018700 | 9301745 | 9301801 | − |
| 196.8 | TACTCAGAGCGGGACTGGAATGT | 383 | GL018700 | 9297778 | 9297834 | − |
| 176.4 | TTGGCTGAGACAGTGTGACAT | 343 | 12 | 148107814 | 148107872 | + |
| 174.9 | TACCTAGAACAGCAACGGAGTGT | 341 | GL018700 | 9249628 | 9249684 | − |
| 150 | TACCTAGAACAGCAACGGAGTGT | 292 | GL018700 | 9259730 | 9259786 | − |
| 135.4 | ACCGCGTCTCCGCTCCCCGCAG | 269 | 15 | 76135699 | 76135759 | − |
| 127.7 | TTCTGGGCAGGCATCGCGCTGG | 254 | GL018928 | 57136 | 57193 | + |
| 125.4 | AGGGGCTGGCACTGTGGCAT | 252 | GL018822 | 301512 | 301571 | + |
| 108.6 | TGTCTCCTGTGCACTTCCCAGG | 219 | 17 | 81223265 | 81223340 | − |
| 72.3 | TGCCTGTGCCCCTTGGGAGTCT | 146 | AAGW02083711 | 3744 | 3803 | − |
| 72.3 | TGCCTGTGCCCCTTGGGAGTCT | 146 | AAGW02083842 | 3863 | 3922 | − |
| 72.3 | TGCCTGTGCCCCTTGGGAGTCT | 146 | GL019111 | 106599 | 106658 | − |
| 72.3 | TGCCTGTGCCCCTTGGGAGTCT | 146 | GL019111 | 136410 | 136469 | − |
| 64.1 | TGCAGGAACAAGCATGTGTAT | 123 | 5 | 36025250 | 36025306 | + |
| 58 | AGGGGCCAGTGCTGTGGTGTAG | 110 | 7 | 13262868 | 13262944 | + |
| 57 | TGTGGGGACCATTGTTGAGTGC | 118 | 17 | 73354107 | 73354166 | − |
| 52.9 | CCGTGACCGAGAGCGAGAACGA | 101 | 19 | 43996541 | 43996610 | + |
| 47.4 | TTCCTGTGCTGTTCTGGGTAG | 90 | GL018700 | 9242143 | 9242199 | − |
| 47.1 | TGACTGCAGTGCCAGTGTCCCAT | 90 | 14 | 153930371 | 153930428 | + |
| 46.3 | TTCTGCCTGTGTAAGGAATGC | 99 | 17 | 11866993 | 11867054 | + |
| 46 | GAAGCGTAGGCGTGCTGGTGG | 94 | AAGW02083711 | 3160 | 3217 | + |
| 45.5 | GAAGCGTAGGCGTGCTGGTGG | 93 | AAGW02083842 | 3276 | 3332 | − |
| 45.5 | GAAGCGTAGGCGTGCTGGTGG | 93 | GL019111 | 106017 | 106073 | − |
| 39 | ATGATTTTCAGCTAAGAATGG | 74 | 7 | 99353953 | 99354012 | − |
| 37.4 | TTCCTGGAACGCTCGGGGAGC | 70 | GL018725 | 923382 | 923437 | − |
| 37.1 | AGGGGCTGGCACTGTGGCAC | 80 | GL019312 | 53901 | 53961 | − |
| 35.8 | TGGAACCAGGACCTGCGGGGGC | 66 | AAGW02082842 | 67 | 727 | − |
| 32.7 | TTGGGACTGGTGCTGCGACG | 61 | 2 | 83442936 | 83442996 | − |
| 32.4 | AGGGACTGGCTCTGTGGCG | 69 | 12 | 87153697 | 87153757 | − |
| 30.6 | TGGCCTGCAGTGCTGGCATCCT | 58 | 19 | 31771015 | 31771069 | + |
| 29.6 | TAAGGGCTGGCGATGTGGCAT | 55 | GL018717 | 1575634 | 1575696 | + |
| 26.6 | TCACCACCAAGGAACCAACCAC | 48 | 4 | 35492486 | 35492547 | + |
| 24.5 | TACCTAGAACAGCAATGGAGTGT | 49 | GL018700 | 9263075 | 9263130 | − |
| 22.5 | GAAGGTGGCCTGCCCTGTTCT | 41 | GL019134 | 104104 | 104165 | − |
| 21.3 | ACACAACATTGAAGGAGGAAT | 52 | 19 | 25357266 | 25357324 | + |
| 21 | CACCTGCAGTGCCGGCATCCCAT | 40 | 17 | 49336057 | 49336114 | + |
| 20.4 | TTGGGGCTGGCACTGTGGCAT | 38 | 17 | 8009414 | 8009472 | + |
| 17.9 | CGCAGGTCTGGGGTCTGAGCTGC | 32 | GL019162 | 55775 | 55837 | − |
| 17 | TGCAGTATGGGCCCCGTCCTG | 30 | GL019015 | 38527 | 38585 | + |
| 16.7 | CCACCTGCAGTGCTGGCATCCC | 30 | 9 | 36322677 | 36322732 | + |
| 16.5 | TACTCAGAGAGGCACTGGCATGT | 29 | GL018700 | 9301076 | 9301132 | − |
| 16.2 | ACGGTCCTCTGCCCACTCAG | 36 | 9 | 99528136 | 99528196 | + |
| 15.4 | TGGGAGACAGCAGAGAGAGGCT | 28 | 2 | 130747420 | 130747482 | + |
| 15 | AGGGGTGAGCCCCACAGAGTCT | 35 | 12 | 20787416 | 20787486 | − |
| 14.3 | CTTGCCTTTGCACTTGGGGGTT | 24 | X | 108854306 | 108854365 | − |
| 13.3 | TTGACCGAGGATCTGGCCCCAG | 23 | 13 | 138301007 | 138301069 | + |
| 12.5 | TTCTAGCAGTGCCGGCACCCCAT | 22 | 11 | 65211194 | 65211252 | + |
| 12.5 | GCGGAGCAGGTCTTGGATCAT | 21 | 19 | 19424456 | 19424516 | − |
| 12.4 | GATGGGAGTCAGTGGCCGTGG | 21 | 21 | 12776073 | 12776128 | + |
| 12.3 | TGAGAGTGGGTTCAAGTC | 33 | 7 | 122613332 | 122613382 | − |
| 12.2 | CAAGGCTGGGAGATGCTCTGA | 21 | 10 | 10979171 | 10979231 | − |
| 12.2 | CACCTGCAGTGCCGGCATCCCAT | 31 | 12 | 45037872 | 45037930 | − |
| 12.1 | TGCATCACATTGCCAGTCCTGA | 21 | 10 | 16906372 | 16906438 | − |
| 11.3 | CTTGGCTGTGGCTGCCCCAGGTTC | 27 | 2 | 83422922 | 83422981 | − |
| 11.3 | ACCACACGGGGCACCGCCGGGCTGG | 26 | GL018914 | 202622 | 202684 | + |
| 10.9 | TGGGGCTGCCTGCCATGTGTGG | 26 | AAGW02083711 | 4853 | 4907 | − |
| 10.1 | TTGAGGGCATCTTAAGAGAGGA | 19 | 1 | 190847846 | 190847913 | + |
| 9.6 | CAAAAGTTCATGGAAGTGGAAG | 16 | 14 | 104692557 | 104692613 | + |
| 7.3 | GGGCTGTATGTGTGCTTTTGC | 13 | 1 | 70076462 | 70076522 | − |
| 7.2 | GGGGCTGGCGCTGTGGTGCAG | 11 | 7 | 166406071 | 166406125 | − |
| 7.2 | AGGGCCGGCACTGTAGTATAGT | 11 | X | 47659871 | 47659926 | + |
| 7.2 | AGACAGGAAGGGTGTGTCTGT | 11 | GL019171 | 65160 | 65234 | − |
| 6.9 | CAATCAGCCTACTGGGAACAT | 26 | 1 | 123190047 | 123190103 | + |
| 6.8 | TCTGTCTGCCCTCCGCTTGCT | 10 | 9 | 7084950 | 7085008 | − |
| 6.7 | ACCTGCATGTTAGTCTAATGTTCTG | 11 | 4 | 77890296 | 77890385 | + |
| 6.3 | CCCACCCACCCCTGTGAGATCG | 8 | GL018940 | 47229 | 47287 | + |
| 5.3 | TGACCCCCGGCCGGACCCCAC | 6 | 4 | 84827251 | 84827317 | − |
| 5.2 | AAGGGATCGGCATTCTGGCAT | 9 | 1 | 184713261 | 184713319 | − |
| 5.2 | AGTCCAGGGCTTAGTCAGCGG | 7 | 15 | 50955589 | 50955648 | + |
| 5 | TCGTATCCACCTCTGTTGCAG | 19 | GL018831 | 47863 | 47943 | + |
| 5 | ATCATGTATGATACTGCACACA | 14 | GL019074 | 61059 | 61121 | + |
| 4.5 | TATTCAAGCCCAGCGGAGGTAC | 5 | 2 | 159928226 | 159928285 | + |
| 4.5 | TATGTTTGCTGTTTTGTGAGAC | 10 | 7 | 172929928 | 172929992 | − |
| 4.4 | CCCCCAAGGACACTGCCTGCCTAC | 5 | 10 | 45287368 | 45287427 | + |
| 4.4 | TAGGGAGAGAATGAGTGGACTGA | 8 | 20 | 30157180 | 30157238 | + |
| 4.2 | GTCGCACGCACGCCCGCCTCGG | 12 | 1 | 130454171 | 130454237 | − |

SEQ ID NOS: 98-133 are present in Table S2 below.

SUPPLEMENTARY TABLE S2
List of novel miRNAs in Exo-A.

| | | Total | | | | |
|---|---|---|---|---|---|---|
| miRDeep | Consensus Mature | read | | | | |
| 2 score | sequence | counts | Chromosome | Start | Stop | Strand |
| 64482 | GGTGCTGTAGGCTTTAAA | 126510 | 19 | 54756247 | 54756283 | + |
| 486.7 | GAAGTCGCAGCTCTCTGGGC | 951 | 14 | 163848014 | 163848071 | − |
| 282.1 | GTCTGAGCGGCGGCCACT | 559 | 13 | 110485608 | 110485664 | + |
| 251.8 | TGTCTCCTGTGCACTTCCCAGG | 500 | 17 | 81223265 | 81223340 | − |
| 220.8 | TCAAAAAGTCGGGGGGAAATGC | 430 | GL018776 | 1023380 | 1023447 | − |
| 70.2 | CCGTGACCGAGAGCGAGAACGA | 135 | 19 | 43996541 | 43996610 | − |
| 62.9 | TTCCTGTGCTGTTCTCGGTAG | 121 | GL018700 | 9256430 | 9256486 | − |
| 62.9 | TTCCTGTGCTGTTCTCGGTAG | 121 | GL018700 | 9286959 | 9287015 | − |
| 47.7 | TCAAAGGAAGAAGACTGGCGT | 91 | 11 | 3609112 | 3609171 | + |
| 44.9 | TTGGCTGAGACAGTGTGACAT | 85 | 12 | 148107814 | 148107871 | + |
| 25.8 | CAATCAGCCTACTGGGAACAT | 63 | 1 | 123190047 | 123190103 | + |
| 20.6 | AGGGGTGAGCCCCACAGAGTCT | 46 | 12 | 20787417 | 20787486 | − |
| 17.5 | TGGGCCCACAGAGACTCTGG | 31 | GL018897 | 256545 | 256602 | - |
| 17.4 | TTCCTGTGCTGCTCTGAGTAG | 40 | GL018700 | 9249628 | 9249684 | + |
| 17.3 | CCTAGAGTGTCGGCATCCCTTT | 40 | GL018822 | 301512 | 301571 | + |
| 16.8 | TTCTGCCTGTGTAAGGAATGC | 41 | 17 | 11866993 | 11867054 | + |
| 15.7 | ATCATGTATGATACTGCACACA | 35 | GL019074 | 61059 | 61121 | + |
| 13.7 | TGTGGGGACCATTGTTGAGTGC | 33 | 17 | 73354107 | 73354166 | + |
| 13.3 | CGCAGGTCTGGGGTCTGAGCTGC | 23 | GL019162 | 55776 | 55837 | − |
| 13 | ATGATTTTCAGCTAAGAATGG | 23 | 7 | 99353950 | 99354012 | − |
| 11.6 | GTGTATGATGACTGGATT | 20 | GL018725 | 3255292 | 3255368 | + |
| 10.9 | GCGGAGCAGGTCTTGGATCAT | 18 | 19 | 19424457 | 19424516 | - |
| 10.8 | TACTCAGAGCGGGACTGGAATGT | 18 | GL018700 | 9301745 | 9301801 | − |
| 10.7 | TACTCAGAGCGGGACTGGAATGT | 18 | GL018700 | 9297778 | 9297834 | − |
| 9.9 | CATCCAGAAGTCCAGGGACTTT | 20 | GL018705 | 625149 | 625204 | + |
| 9.8 | TTCCTGGAACGCTCGGGGAGC | 16 | GL018725 | 923381 | 923437 | − |
| 8.8 | TACCTAGAACAGCAACGGAGTGT | 15 | GL018700 | 9259730 | 9259786 | − |
| 6.9 | TGAACCTGTGATGCCAGCATCC | 10 | 8 | 33724831 | 33724887 | − |
| 6 | TATGTTTGCTGTTTTGTGAGAC | 13 | 7 | 172929928 | 172929992 | − |
| 6 | CTTCCTGGCTGACGTGGAGTCC | 8 | 20 | 9171823 | 9171886 | + |
| 5.7 | GGCAGGGCCTGCTTCCTTCC | 8 | GL019134 | 104104 | 104165 | − |
| 5.2 | CGTCTGGGCTGGCCCGAGCG | 14 | GL019068 | 92002 | 92058 | − |
| 5.1 | TACCTAGAACAGCAATGGAGTGT | 7 | GL018700 | 9242142 | 9242199 | − |
| 5 | CCTCTGCACTCTCTCTCACCAG | 14 | GL018821 | 265484 | 265554 | − |
| 4.4 | TCTGGCTTCAAGGTGCTGTGT | 5 | GL018725 | 2252226 | 2252285 | − |
| 4.1 | ACACAGCAGACTCAGAGG | 9 | 14 | 122355007 | 122355074 | + |

### Discussion

Demonstrated herein is the therapeutic use of characterized exosomes to stimulate cell homing and angiogenic differentiation for pulp regeneration. Exosomes were isolated from DPSCs cultured under a growth or angiogenic differentiation condition for Exo-G and Exo-A, respectively. The results revealed that both DPSC-Exos significantly promoted cell proliferation (FIG. 2B), migration (FIG. 2C, D), and angiogenic differentiation (FIG. 3) when treated with 5×10⁸/mL exosomes. In NGS, we profiled miRNAs encapsulated in DPSC-Exos to identify key miRNAs for cell homing and angiogenesis (FIG. 4, Tables 1 and 2).

DPSC-Exos were isolated by a polymer-based precipitation method which has the advantages of saving time and labor, being easily scalable, and having a higher yield compared to ultracentrifugation [31]. According to a standard guideline from the International Society of Extracellular Vesicles (ISEV) [32], isolated exosomes were characterized in terms of size distribution and concentration, exosome-specific protein markers, morphology, and cellular uptake. In NTA, both DPSC-Exos showed a similar size distribution with a peak at approximately 104 nm, whereas Exo-G had an approximately two-fold enrichment in the concentration compared to Exo-A (FIG. 1E). This difference can be explained by different cell types which affect exosome yield. Although there was no direct comparison between DPSCs and angiogenic differentiated DPSCs (or endothelial cells) in the previous study, it is known that MSCs generate a scalable yield of exosomes [33,34]. Exosomes consist of various cargoes, and proteins, especially, in the membrane and cytosol are used for exosome markers. Herein, we validated the exosome compositions including tetraspanins (CD63 and CD81), endosomal sorting complex required for transport (ESCRT) (TSG101 and ALIX), and membrane transport and fusion (ANXA5 and FLOT1) using an antibody array (FIG. 1F) [35]. The spheroid morphology of exosomes was confirmed in SEM, and the size distribution (˜100 nm) was consistent with the result of NTA (FIG. 1G, H). Moreover, exosomes successfully internalized into recipient DPSCs within 48 h of exposure (FIG. 11, J).

One appeal of this DPSC-Exo-based cell homing strategy lies in their ability to heal by self-congregating at exposure sites. Cell homing or migration, which is a prerequisite process during tissue regeneration, is defined as a directional cell movement in response to chemoattractant and is important in both the development and maintenance of multicellular organisms. Recently, exosomes have been shown to play a role in cell migration due to the function of directional sensing, leader-follower behavior, cell adhesion, and extracellular matrix (ECM) degradation [36,37]. Specifically, exosomes contain cyclic adenosine monophosphate (cAMP) that can be actively synthesized and released to promote chemotaxis [38]. Sung and colleagues have reported that migrating cells leave stationary exosome trails to be used as paths for follower cells [39]. Herein, pHluo_M153R-CD63-positive exosome trails were observed behind cells, and following cells exhibited pathfinding behavior on the trails. In addition, cell adhesion molecules, such as integrins and fibronectin highly contained in exosomes, regulate cell migration speed [40]. Lastly, cell migration or invasion is highly achieved by proteolytic ECM degradation to allow for a cell path, and exosomes carry membrane-linked matrix metalloproteinases (MMPs) to promote the invasive motility of cells across ECM [41,42].

Exosomes or conditioned medium cultured from lineage-specific differentiated human bone marrow-derived stromal cells (HMSCs) and DPSCs have use in odontogenesis. Hu and colleagues have shown that exosomes isolated under an odontogenic differentiation conditions induced dramatic increases of odontogenic marker genes such as bone morphogenetic protein 9 (BMP9), alkaline phosphatase (ALP), Runt-related transcription factor 2 (RUNX2), and type I collagen [28,43]. Herein, we compared the effects of Exo-A and Exo-G on activities related to tissue regeneration. However, despite differences in miRNA content, there were almost no differences between Exo-G and Exo-A in terms of effects on DPSC proliferation, migration, and angiogenic differentiation.

Previous studies have reported that the conditioned medium and lysates of human DPSCs promoted angiogenesis by releasing angiogenic factors such as VEGF and monocyte chemotactic protein-1 (MCP-1) [44,45]. Regardless of a similar trend in the results, distinct levels of exosomal miRNA expression were identified in the comparison between Exo-G and Exo-A. Several mature miRNAs (ocu-miR-205-5p [46], ocu-let-7i-3p [47], ocumiR-874-3p [48], and ocu-miR-29a-3p [47,49,50]) previously shown to be angiogenic were significantly over-represented in Exo-A versus Exo-G (Table 1). In contrast, ocu-miR-503-5p and ocu-miR-20b-5p, which were under-represented in Exo-A compared to Exo-G, may have allowed higher levels of CD40 [51] and hypoxia-inducible factor 1 (HIF-1) expression [52] in Exo-A treated cultures, which could also lead to greater angiogenic stimulation.

miRNAs are small non-coding regulatory RNAs and play a role in post-transcriptional regulation of gene expression either by promoting messenger RNA (mRNA) degradation or by blocking the translation of transcribed sequences. Rather than targeting a specific gene, a single miRNA can regulate multiple pathways by modulating more than one Mrna [54]. miRNAs are secreted and transferred to target recipient cells via exosomes, thereby leading to functional cellular changes [55]. Herein, the profiles of miRNAs in DMSC-Exos strongly advocate for their potential in pulp regeneration. Both Exo-G and Exo-A contain mature (over 450), hairpin (over 250), and novel miRNAs (FIG. 4, Tables S1 and S2). Among mature miRNA sequences, ocu-miR-199a-3p, ocu-miR-221-3p, ocu-miR-24-3p, and ocu-miR-21-5p were counted in over 100,000 total reads in both exosomes (FIG. 4A-C). miR-199a-3p functions as a redundant network of the nitric oxide synthase (NOS)/nitric oxide (NO) pathway, thereby inducting endothelial tubulogenesis and cardiac regeneration [56,57]. The function of miR-221-3p is controversial in terms of angiogenesis. As an anti-angiogenic miRNA, it inhibits cell proliferation and migration in endothelial cells [58]. On the other hand, miR-221 is a regulatory node during angiogenesis [59]. Ocu-miR-24-3p exacted from adipose-derived mesenchymal stem cells can accelerate corneal epithelial migration in vitro and in vivo [60]. Lastly, miR-21-5p has a role in regulating cell migration by ECM degradation and promoting angiogenesis via the extracellular signal-regulated kinase (ERK)/mitogen-activated protein kinase (MAPK) signal pathway [61,62]. Hence, targeting its associated miRNA(s) can serve as a novel therapeutic armamentarium for a given pathophysiologic condition including pulp damage. miRNA sequences can be loaded in engineered exosomes for clinical application, thereby effectively replacing DPSC cultures as a potential means of exosome production.

Most miRNAs are transcribed by RNA polymerase II to generate primary miRNAs (pri-miRNAs), which are then processed into hairpin precursor miRNAs (pre-miRNAs) via Drosha, a type III ribonuclease, resulting in mature miRNAs. Herein, we compared levels of hairpin miRNAs in DPSC-Exos from cells cultured under angiogenic or growth conditions (Exo-A versus Exo-G). A total of 291 hairpin miRNAs were found in DPSCExos. Among these, levels of 18 hairpin miRNAs were significantly different in the two groups (Table 2). Seven of these (ocu-miR-708, ocu-miR-134, ocu-miR-125b-2, ocu-miR-140, ocu-miR-29b-1, and ocu-miR-214) were present at significantly higher levels in Exo-A than in Exo-G, and five (ocu-miR-574, ocu-miR-503, ocu-miR-30a, ocu-miR-146a, and ocumiR-671) were significantly lower in Exo-A versus Exo-G in both mature and hairpin miRNAs. Several of the miRNAs up-regulated in Exo-A have been strongly associated with tissue regeneration. For example, miR-708 was shown to protect against stress-induced apoptosis and promote myocardial regeneration in an animal study of injured hearts [63]. Sun and colleagues have also shown that miR-140-5p regulated DPSC proliferation and differentiation via the toll-like receptor 4 (TLR-4) [64].

RT-PCR revealed a positive effect of DPSC-Exos on angiogenic marker expression only when angiogenic growth factors (fibroblastic growth factor B, VEGF, insulin-like growth factor-1, and epidermal growth factor) were present in the culture medium (FIG. 3). Under these conditions, the expression of the angiogenic markers, VEGFA, FLT1, and PECAM1 was up-regulated up to four-fold by both Exo-G and Exo-A. In contrast, there was no effect of exosomes on cells cultured in basal medium lacking angiogenic growth factors, implying that exosomes may potentiate the effects of these factors.

### Conclusions

Provided herein are the effects of stem cell-derived exosomes on cell homing and angiogenic lineage-specific differentiation for pulp regeneration. The results show that DPSC-Exos (both Exo-G and Exo-A) significantly promoted cell proliferation, migration, and angiogenic differentiation although there was no superior effect of Exo-A on angiogenic differentiation compared to Exo-G in this in vitro study. In addition, we identified key miRNAs which regulate cell homing and angiogenesis. Therefore, the exosome-based cell homing and angiogenic differentiation strategy has application dental pulp regeneration.

### Example II

### DPSC-Exo-O

Exosomes were isolated from rabbit DPSCs cultured under odontogenic differentiation condition. For Exo-O isolation, the cells were cultured in an odontogenic differentiation medium for 10 days. The odontogenic induction medium (ScienCell, Cat #7531; sciencellonline.com/mesenchymal-stem-cell-osteogenic-differentiation-medium/. MODM consists of 500 ml of basal medium, 25 ml of fetal bovine serum (FBS, Cat. #0025), 5 ml of Mesenchymal Stem Cell Osteogenic Differentiation Supplement (MODS, Cat. #7532), and 5 ml of penicillin/streptomycin solution (P/S, Cat. #0503).) was replaced with α-MEM growth medium containing 10% exosome-depleted FBS, and the conditioned medium (CM) was collected after 48 h. DPSC-Exos were isolated by a precipitation method using ExoQuick-TC™ (System Biosciences, Palo Alto, CA, USA). In brief, CM was centrifuged at 3000×g for 15 min to remove cells and cell debris. The supernatant was mixed with an ExoQuick-TC™ solution at 4° C. overnight. After centrifugation, the exosome pellet was resuspended in PBS and stored at 80° C. before use.

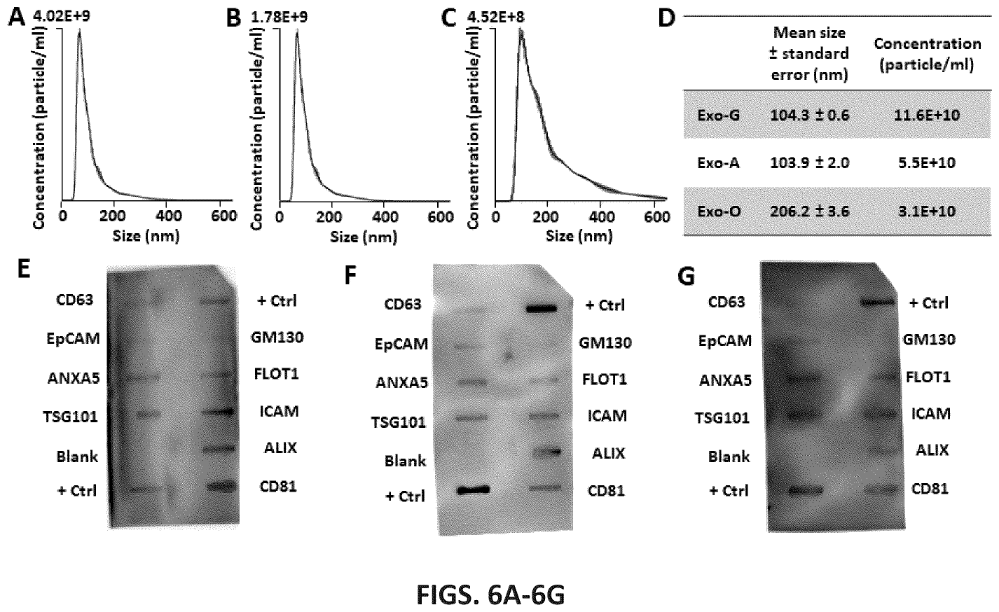

Characterization of DPSC-Exo-O: The characterization of exosomes was confirmed by nanoparticle tracking analysis (NTA) and Exo-Check™ exosome antibody array. Mean size of particles was the biggest in Exo-O (206.2 nm versus 104.3 nm Exo-G and 103.9 nm Exo-A), and the concentration of Exo-G (11.6×10¹⁰ particles/ml) was 2-3 times higher than Exo-A (5.5×10¹⁰ particles/ml) and Exo-O (3.1×10¹⁰ particles/ml) (FIG. 6A-D). All exosomes showed positive expression of ANXA5, TSG101, FLOT1, ICAM, ALIX, and CD81, and negative expression of GM130 as a negative marker (FIG. 6E-G).

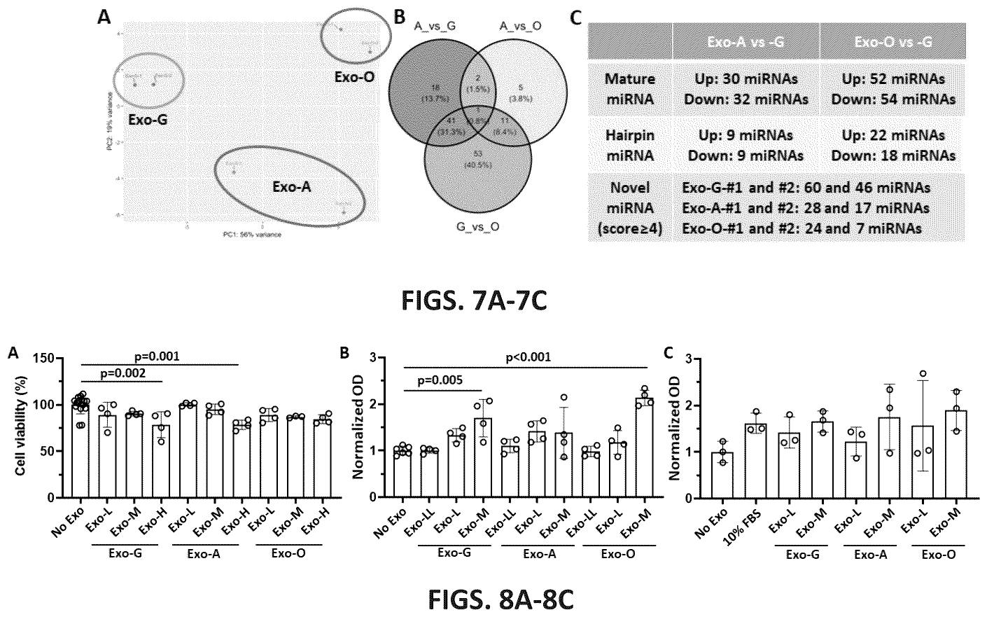

Exosomal miRNA profile via Next-generation sequencing (NGS): Two batches of exosomes (Exo-G, Exo-A, and Exo-0) were processed for total RNA isolation using the SeraMir Exosome RNA Purification Column kit according to the manufacturer's instructions. Small RNA libraries were designed, and the purified library was quantified with High Sensitivity DNA Reagents and High Sensitivity DNA Chips. The top 5 high-abundance miRNAs were ocu-miR-146a-5p (negative regulation of inflammatory response), ocu-miR-199a-3p (positive regulation of endothelial cell migration), ocu-miR-122-5p (stimulates the proliferation and DNA synthesis of spermatogonial stem cells), ocu-miR-221-3p (positive regulation of epithelial cell migration), and ocu-miR-143-3p (inhibits angiogenic and odontogenic differentiation) in Exo-G, ocu-miR-221-3p, ocu-miR-21-5p (enhances angiogenesis and odontogenesis), ocu-miR-199a-3p, ocu-miR-24-3p (inhibits angiogenesis), and ocu-miR-23b-3p (potential regulators of angiogenesis) in Exo-A, and ocu-miR-21-5p, ocu-miR-199a-3p, ocu-miR-221-3p, ocu-miR-24-3p, and ocu-miR-214-3p (inhibits odontogenesis) in Exo-O. Each group showed good clustering in principal component analysis (PCA) (FIG. 7A). We found significantly up- or down-expressed miRNAs in Exo-A and Exo-O when compared with Exo-G (FIG. 7B). Among the up-expressed miRNAs, miR-205-5P, miR-29c-3p, miR-29a-3p, miR-30b-5p, and miR-let-7i can promote angiogenesis, and miR-29b-2-5p and miR-let-7i-3p contribute to positive regulation of osteoblast differentiation. In addition, we obtained the list of up-/down-expressed hairpin and novel miRNAs (FIG. 7C and Table 4).

TABLE 4
List of miRNA expression (top 20)

| | Total Reads | | Total Reads | | Total Reads |
|---|---|---|---|---|---|
| Exo-G | Count | Exo-A | Count | Exo-O | Count |
| ocu-miR-146a-5p | 296,149.6 | ocu-miR-221-3p | 240,802.4 | ocu-miR-21-5p | 320,322.8 |
| ocu-miR-199a-3p | 217,998.9 | ocu-miR-21-5p | 192,376.3 | ocu-miR-199a-3p | 197,071.3 |
| ocu-miR-122-5p | 190,513.3 | ocu-miR-199a-3p | 184,859.6 | ocu-miR-221-3p | 188,002.2 |
| ocu-miR-221-3p | 185,003.3 | ocu-miR-24-3p | 180,369.2 | ocu-miR-24-3p | 90,800.2 |
| ocu-miR-143-3p | 143,493.5 | ocu-miR-23b-3p | 83,437.1 | ocu-miR-214-3p | 86,211.2 |
| ocu-miR-24-3p | 137,071.1 | ocu-miR-214-3p | 83,163.6 | ocu-miR-23b-3p | 70,641.3 |
| ocu-miR-21-5p | 122,765.1 | ocu-miR-143-3p | 55,696.6 | ocu-miR-92a-3p | 67,659.8 |
| ocu-miR-92a-3p | 61,986.4 | ocu-miR-29a-3p | 50,879.1 | ocu-miR-29a-3p | 65,430.4 |
| ocu-miR-23b-3p | 49,175.0 | ocu-miR-122-5p | 50,631.5 | ocu-miR-143-3p | 63,079.2 |
| ocu-miR-27b-3p | 47,342.7 | ocu-miR-27b-3p | 48,073.3 | ocu-miR-22-3p | 58,819.7 |
| ocu-miR-214-3p | 42,716.4 | ocu-miR-92a-3p | 46,452.7 | ocu-miR-27b-3p | 47,713.5 |
| ocu-let-7a-5p | 38,945.8 | ocu-miR-146a-5p | 44,604.0 | ocu-miR-125b-5p | 38,903.8 |
| ocu-miR-16b-5p | 38,384.9 | ocu-miR-125b-5p | 36,816.5 | ocu-miR-34a-5p | 27,086.6 |
| ocu-miR-378-3p | 38,061.7 | ocu-miR-22-3p | 36,812.6 | ocu-let-7i-5p | 24,071.4 |
| ocu-miR-16a-5p | 35,936.5 | ocu-let-7i-5p | 21,074.8 | ocu-miR-423-5p | 21,867.3 |
| ocu-miR-34a-5p | 34,561.7 | ocu-miR-152-3p | 19,322.4 | ocu-miR-16a-5p | 20,585.0 |
| ocu-miR-93-5p | 31,710.0 | ocu-let-7a-5p | 18,439.0 | ocu-miR-146a-5p | 20,118.2 |
| ocu-miR-152-3p | 28,732.5 | ocu-miR-378-3p | 18,252.7 | ocu-miR-100-5p | 19,766.9 |
| ocu-miR-100-5p | 27,396.5 | ocu-miR-103a-3p | 16,753.6 | ocu-miR-152-3p | 19,489.9 |
| ocu-let-7b-5p | 25,971.9 | ocu-miR-16a-5p | 16,701.8 | ocu-miR-29c-3p | 18,416.9 |

Effect of exosomes on cell toxicity, proliferation, and migration: In cytotoxicity test, there was no significant cell death in Exo-O, however, the viability was significantly decreased in 5×10′ Exo-G and Exo-A (FIG. 8A). In all groups, exosomes induced slight increase of cell proliferation at day 3 (FIG. 8B). Cell migration was enhanced in 5×10⁸/ml Exo-O compared to no exosome control.

Effect of exosomes on odontogenic differentiation: The effect of odontogenesis was examined by alkaline phosphatase (ALP) assay. Exo-A and Exo-O at 5×10⁸/ml concentration was treated in DPSCs and the conditioned media was harvested for ALP measurement. Both Exo-G and Exo-O induced significant increase of ALP as a marker of odontogenesis (FIG. 9).

### BIBLIOGRAPHY

1. Islam, B.; et al. Med. Sci. Monit. 2007, 13, RA196-RA203.
2. Foreman, P. C.; Int. Endod. J. 1990, 23, 283-297.
3. Mass, E.; Zilberman, U. Quintessence Int. 2011, 42, 547-554.
4. Parirokh, M.; Torabinejad, M. J. Endod. 2010, 36, 16-27.
5. Torabinejad, M.; Parirokh, M. J. Endod. 2010, 36, 190-202.
6. Desai, S.; Chandler, N. A review. J. Endod. 2009, 35, 475-480.
7. Parirokh, M.; Torabinejad, M. J. Endod. 2010, 36, 400-413.
8. Ghoddusi, J.; Forghani, M.; Parisay, I. Iran Endod. J. 2014, 9, 15-22.
9. Ward, J. Aust. Endod. J. 2002, 28, 29-37.
10. Iohara, K.; et al. Tissue Eng. Part A 2011, 17, 1911-1920.
11. Ishizaka, R.; et al. Biomaterials 2012, 33, 2109-2118.
12. Xuan, K.; et al. Sci. Transl. Med. 2018, 10, eaaf3227.
13. Mao, J. J.; et al. Dent. Clin. N. Am. 2012, 56, 639-649.
14. Eramo, S.; Natali, A.; Pinna, R.; Milia, E. Int. Endod. J. 2018, 51, 405-419.
15. Huang, G. T.; Gronthos, S.; Shi, S. J. Dent. Res. 2009, 88, 792-806.
16. Shi, X.; Mao, J.; Liu, Y. Stem Cells Transl. Med. 2020, 9, 445-464.
17. Goldberg, M.; et al. Pharmacol. Res. 2008, 58, 137-147.
18. Lai, R. C.; et al. Stem Cell Res. 2010, 4, 214-222.
19. Tao, S. C.; et al. Theranostics 2017, 7, 180-195.
20. Tetta, C.; et al. Muscles Ligaments Tendons J. 2012, 2, 212-221.
21. Zhang, B.; et al. Stem Cells 2015, 33, 2158-2168.
22. Zhang, S.; et al. Osteoarthr. Cartil. 2016, 24, 2135-2140.
23. Raposo, G.; Stoorvogel, W. J. Cell Biol. 2013, 200, 373-383.
24. He, L.; Zhang, H. Stem Cell Rev. Rep. 2019, 15, 3-12.
25. Tao, S. C.; Guo, S. C.; Zhang, C. Q. Int. J. Biol. Sci. 2017, 13, 828-834.
26. Guo, S. C.; et al. Theranostics 2017, 7, 81-96.
27. Zhang, S.; et al. Biomaterials 2018, 156, 16-27.
28. Hu, X.; et al. Stem Cell Res. Ther. 2019, 10, 170.
29. Ivica, A.; et al. J. Clin. Med. 2020, 9, 491.
30. Stanko, P.; et al. Stem Cells Int. 2018, 2018, 8973613.
31. Coughlan, C.; et al. Curr. Protoc. Cell Biol. 2020, 88, e110.
32. Thery, C.; et al. J. Extracell. Vesicles 2018, 7, 1535750.
33. Gurunathan, S.; et al., Cells 2019, 8, 307.
34. Yeo, R. W.; et al. Adv. Drug Deliv. Rev. 2013, 65, 336-341.
35. Gurung, S.; et al. Cell Commun. Signal. 2021, 19, 47.
36. Sung, B. H.; Parent, C. A.; Weaver, A. M. Dev. Cell 2021, 56, 1861-1874.
37. Maas, S. L. N.; Breakefield, X. O.; Weaver, A. M. Trends Cell Biol. 2017, 27, 172-188.
38. Kriebel, P. W.; et al. J. Cell Biol. 2018, 217, 2891-2910. [
39. Sung, B. H.; et al. Nat. Commun. 2020, 11, 2092.
40. Jimenez, L.; et al. Proteome Res. 2019, 18, 947-959.
41. Hakulinen, J.; et al. J. Cell Biochem. 2008, 105, 1211-1218.
42. Laghezza Masci, V.; et al. J. Circ. Biomark. 2016, 5, 1849454416663660.
43. Narayanan, R.; et al. Stem Cells Int. 2016, 2016, 3808674.
44. Bronckaers, A.; et al. PLoS ONE 2013, 8, e71104.
45. Hilkens, P.; et al. Stem Cell Res. 2014, 12, 778-790.
46. Sun, L. L.; et al. J. Cell Mol. Med. 2019, 23, 8493-8504.
47. Brunello, G.; et al. Pharmaceutics 2022, 14, 908.
48. Xie, K.; et al. Am. J. Physiol. Cell Physiol. 2020, 319, C579-C588.
49. Lu, G. D.; et al. Front. Cell Dev. Biol. 2020, 8, 608521.
50. Yang, Z.; et al. Biochem. Biophys. Res. Commun. 2013, 434, 143-149.
51. Lee, A.; et al. Sci. Rep. 2017, 7, 2528.
52. Lei, Z.; et al. PLoS ONE 2009, 4, e7629.
53. Ajay Sharma, L.; et al. J. Appl. Biomater. Funct. Mater. 2017, 15, e244-e250.
54. Bader, A. G.; Brown, D., Winkler, M. Cancer Res. 2010, 70, 7027-7030.
55. Valadi, H.; et al. Nat. Cell Biol. 2007, 9, 654-659.
56. Eulalio, A.; et al. Nature 2012, 492, 376-381.
57. Joris, V.; et al. Arterioscler. Thromb. Vasc. Biol. 2018, 38, 2345-2357.
58. Urbich, C.; et al. Cardiovasc. Res. 2008, 79, 581-588.
59. Nicoli, S.; et al. Dev. Cell 2012, 22, 418-429.
60. Sun, X.; et al. Bioact. Mater. 2022; in press.
61. Ma, S.; et al. Arthritis Res. Ther. 2020, 22, 99.
62. Xie, J.; et al. Front. Pharmacol. 2022, 13, 828627.
63. Deng, S.; et al. Theranostics 2017, 7, 1953-1965.
64. Sun, D. G.; et al. Eur. J. Oral Sci. 2017, 125, 419-425.
65. Yin, K.; Wang, S.; Zhao, R. C. Biomark. Res. 2019, 7, 8.
66. Zayed, M.; et al. Stem Cell Res. Ther. 2021, 12, 302.
67. Livak, K. J.; Schmittgen, T. D. Methods 2001, 25, 402-408.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to treat a subject for a dental pulp injury, disease or disorder comprising administering to a subject in need thereof a composition comprising exosomes obtained from dental pulp stem cells (DPSCs) or a composition comprising lipid nanoparticles.

2. A method to regenerate dental pulp comprising administering to a subject in need thereof a composition comprising exosomes obtained from dental pulp stem cells (DPSCs).

3. A method to treat a subject for a dental pulp injury, disease or disorder or to regenerate dental pulp comprising administering to a subject in need thereof a composition comprising engineered exosomes.

4. The method of claim 1, wherein the exosomes are isolated from DPSCs cultured in growth media (DPSC-Exo-G), angiogenic differentiation media (DPSC-Exo-A), odontogenic differentiation media (DPSC-Exo-O) or a combination thereof.

5. The method of claim 1, wherein the exosomes or lipid nanoparticles display CD63, EpCAM, ANXA5, TSG101, FLOT1, ICAM, ALIX, CD81 or a combination thereof.

6. The method of claim 4, wherein the exosomes have a mean particle size about 80 nm to about 250 nm.

7. The method of claim 1, wherein the composition comprises lipid nanoparticles.

8. The method of claim 6, wherein the mean particle size is about 104 nm.

9. The method of claim 6, wherein the mean particle size is about 206 nm.

10. The method of claim 1, wherein the exosomes or lipid nanoparticles comprise a nucleic acid sequence selected from the group consisting of a nucleic sequence of Table 1, Table 2, Table 3, Table 4, a nucleic acid sequence having at least about 90% sequence identity thereto and retaining activity associated with the miRNA or a combination thereof

11. The method of claim 1, wherein the exosomes or lipid nanoparticles comprise a nucleic acid sequence selected from the group consisting of SEQ ID NOs: 1 to 9 or a nucleic acid sequence having at least about 90% sequence identity thereto and retaining activity associated with the miRNA or a combination thereof.

12. The method of claim 1, wherein the composition comprises a nucleic acid sequence selected from the group consisting of SEQ ID NOs: 1 to 9 or a nucleic acid sequence having at least about 90% sequence identity thereto and retaining activity associated with the miRNA or a combination thereof.

13. The method of claim 10, where the miRNA is present in a vector comprising a transcribable nucleic acid molecule encoding the miRNA operably linked to a promoter.

14. Them method of claim 10, wherein the miRNA is independently present in the composition or contained with the exosome.

15. The method of claim 1, wherein the composition further comprises hydrogel comprising fibrinogen and hyaluronic acid (HA).

16. The method of claim 1, wherein the exosomes or lipid nanoparticles are encapsulated in a hydrogel.

17. The method of claim 15, wherein the hydrogel comprises about 12.5% (w/v) fibrinogen and about 0.25% (w/v/) hyaluronic acid (HA).

18. The method of claim 1, wherein the subject is a mammal.

19. The method of claim 18, wherein the mammal is a human.

20. The method of claim 1, wherein the exosomes are isolated from human cells.


SEQUENCE LISTING (133 sequences)

SEQ ID NO: 1 - RNA, 21 nt, synthetic construct
  source 1..21: mol_type = other RNA
agcttatcagactgatgttga

SEQ ID NO: 2 - RNA, 23 nt, synthetic construct
  source 1..23: mol_type = other RNA
atcacattgccagggattaccac

SEQ ID NO: 3 - RNA, 22 nt, synthetic construct
  source 1..22: mol_type = other RNA
tggctcagttcagcaggaacag

SEQ ID NO: 4 - RNA, 22 nt, synthetic construct
  source 1..22: mol_type = other RNA
tggagtgtgacaatggtgtttg

SEQ ID NO: 5 - RNA, 21 nt, synthetic construct
  source 1..21: mol_type = other RNA
tgagatgaagcactgtagctc

SEQ ID NO: 6 - RNA, 22 nt, synthetic construct
  source 1..22: mol_type = other RNA
tgagaactgaattccatgggtt

SEQ ID NO: 7 - RNA, 22 nt, synthetic construct
  source 1..22: mol_type = other RNA
acagtagtctgcacattggtta

SEQ ID NO: 8 - RNA, 22 nt, synthetic construct
  source 1..22: mol_type = other RNA
acagcaggcacagacaggcagt

SEQ ID NO: 9 - RNA, 22 nt, synthetic construct
  source 1..22: mol_type = other RNA
agctacatgtctgctgggtttc

SEQ ID NO: 10 - RNA, 18 nt, synthetic construct
  source 1..18: mol_type = other RNA
gctgtgatggccgagtgg

SEQ ID NO: 11 - RNA, 20 nt, synthetic construct
  source 1..20: mol_type = other RNA
gaagtcgcagctctctgggc

SEQ ID NO: 12 - RNA, 18 nt, synthetic construct
  source 1..18: mol_type = other RNA
ggggatgtagctcagtgg

SEQ ID NO: 13 - RNA, 22 nt, synthetic construct
  source 1..22: mol_type = other RNA
tcaaaaagtcggcgggaaatgc

SEQ ID NO: 14 - RNA, 22 nt, synthetic construct
  source 1..22: mol_type = other RNA
agccagctctgtggcgcagtgg

SEQ ID NO: 15 - RNA, 21 nt, synthetic construct
  source 1..21: mol_type = other RNA
ttcctgtgctgttctcggtag

SEQ ID NO: 16 - RNA, 21 nt, synthetic construct
  source 1..21: mol_type = other RNA
ttcctgtgctgttctcggtag

SEQ ID NO: 17 - RNA, 21 nt, synthetic construct
  source 1..21: mol_type = other RNA
ttcctgtgctgttctcggtag

SEQ ID NO: 18 - DNA, 21 bp, synthetic construct
  source 1..21: mol_type = other DNA
ttcctgtgctgttctcggtag

SEQ ID NO: 19 - RNA, 21 nt, synthetic construct
  source 1..21: mol_type = other RNA
ttcctgtgctgttctcggtag

SEQ ID NO: 20 - RNA, 21 nt, synthetic construct
  source 1..21: mol_type = other RNA
tgggaaccttgggcaggaagt

SEQ ID NO: 21 - RNA, 20 nt, synthetic construct
  source 1..20: mol_type = other RNA
gtgacctgtggtgctggtgg

SEQ ID NO: 22 - RNA, 23 nt, synthetic construct
  source 1..23: mol_type = other RNA
tactcagagcgggactggaatgt

SEQ ID NO: 23 - RNA, 23 nt, synthetic construct
  source 1..23: mol_type = other RNA
tactcagagcgggactggaatgt

SEQ ID NO: 24 - RNA, 21 nt, synthetic construct
  source 1..21: mol_type = other RNA
ttggctgagacagtgtgacat

SEQ ID NO: 25 - RNA, 23 nt, synthetic construct
  source 1..23: mol_type = other RNA
tacctagaacagcaacggagtgt

SEQ ID NO: 26 - RNA, 23 nt, synthetic construct
  source 1..23: mol_type = other RNA
tacctagaacagcaacggagtgt

SEQ ID NO: 27 - RNA, 22 nt, synthetic construct
  source 1..22: mol_type = other RNA
accgcgtctccgctccccgcag

SEQ ID NO: 28 - RNA, 22 nt, synthetic construct
  source 1..22: mol_type = other RNA
ttctgggcaggcatcgcgctgg

SEQ ID NO: 29 - RNA, 20 nt, synthetic construct
  source 1..20: mol_type = other RNA
aggggctggcactgtggcat

SEQ ID NO: 30 - RNA, 22 nt, synthetic construct
  source 1..22: mol_type = other RNA
tgtctcctgtgcacttcccagg

SEQ ID NO: 31 - RNA, 22 nt, synthetic construct
  source 1..22: mol_type = other RNA
tgcctgtgccccttgggagtct

SEQ ID NO: 32 - RNA, 22 nt, synthetic construct
  source 1..22: mol_type = other RNA
tgcctgtgccccttgggagtct

SEQ ID NO: 33 - RNA, 22 nt, synthetic construct
  source 1..22: mol_type = other RNA
tgcctgtgccccttgggagtct

SEQ ID NO: 34 - RNA, 22 nt, synthetic construct
  source 1..22: mol_type = other RNA
tgcctgtgccccttgggagtct

SEQ ID NO: 35 - RNA, 21 nt, synthetic construct
  source 1..21: mol_type = other RNA
tgcaggaacaagcatgtgtat

SEQ ID NO: 36 - RNA, 22 nt, synthetic construct
  source 1..22: mol_type = other RNA
aggggccagtgctgtggtgtag

SEQ ID NO: 37 - RNA, 22 nt, synthetic construct
  source 1..22: mol_type = other RNA
tgtggggaccattgttgagtgc

SEQ ID NO: 38 - RNA, 22 nt, synthetic construct
  source 1..22: mol_type = other RNA
ccgtgaccgagagcgagaacga

SEQ ID NO: 39 - RNA, 21 nt, synthetic construct
  source 1..21: mol_type = other RNA
ttcctgtgctgttctgggtag

SEQ ID NO: 40 - RNA, 23 nt, synthetic construct
  source 1..23: mol_type = other RNA
tgactgcagtgccagtgtcccat

SEQ ID NO: 41 - RNA, 21 nt, synthetic construct
  source 1..21: mol_type = other RNA
ttctgcctgtgtaaggaatgc

SEQ ID NO: 42 - RNA, 21 nt, synthetic construct
  source 1..21: mol_type = other RNA
gaagcgtaggcgtgctggtgg

SEQ ID NO: 43 - RNA, 21 nt, synthetic construct
  source 1..21: mol_type = other RNA
gaagcgtaggcgtgctggtgg

SEQ ID NO: 44 - RNA, 21 nt, synthetic construct
  source 1..21: mol_type = other RNA
gaagcgtaggcgtgctggtgg

SEQ ID NO: 45 - RNA, 21 nt, synthetic construct
  source 1..21: mol_type = other RNA
atgattttcagctaagaatgg

SEQ ID NO: 46 - RNA, 21 nt, synthetic construct
  source 1..21: mol_type = other RNA
ttcctggaacgctcggggagc

SEQ ID NO: 47 - RNA, 20 nt, synthetic construct
  source 1..20: mol_type = other RNA
aggggctggcactgtggcac

SEQ ID NO: 48 - RNA, 22 nt, synthetic construct
  source 1..22: mol_type = other RNA
tggaaccaggacctgcgggggc

SEQ ID NO: 49 - RNA, 20 nt, synthetic construct
  source 1..20: mol_type = other RNA
ttgggactggtgctgcgacg

SEQ ID NO: 50 - RNA, 19 nt, synthetic construct
  source 1..19: mol_type = other RNA
agggactggctctgtggcg

SEQ ID NO: 51 - RNA, 22 nt, synthetic construct
  source 1..22: mol_type = other RNA
tggcctgcagtgctggcatcct

SEQ ID NO: 52 - RNA, 21 nt, synthetic construct
  source 1..21: mol_type = other RNA
taagggctggcgatgtggcat

SEQ ID NO: 53 - RNA, 22 nt, synthetic construct
  source 1..22: mol_type = other RNA
tcaccaccaaggaaccaaccac

SEQ ID NO: 54 - RNA, 23 nt, synthetic construct
  source 1..23: mol_type = other RNA
tacctagaacagcaatggagtgt

SEQ ID NO: 55 - RNA, 21 nt, synthetic construct
  source 1..21: mol_type = other RNA
gaaggtggcctgccctgttct

SEQ ID NO: 56 - RNA, 21 nt, synthetic construct
  source 1..21: mol_type = other RNA
acacaacattgaaggaggaat

SEQ ID NO: 57 - RNA, 23 nt, synthetic construct
  source 1..23: mol_type = other RNA
cacctgcagtgccggcatcccat

SEQ ID NO: 58 - RNA, 21 nt, synthetic construct
  source 1..21: mol_type = other RNA
ttggggctggcactgtggcat

SEQ ID NO: 59 - RNA, 23 nt, synthetic construct
  source 1..23: mol_type = other RNA
cgcaggtctggggtctgagctgc

SEQ ID NO: 60 - RNA, 21 nt, synthetic construct
  source 1..21: mol_type = other RNA
tgcagtatgggccccgtcctg

SEQ ID NO: 61 - RNA, 22 nt, synthetic construct
  source 1..22: mol_type = other RNA
ccacctgcagtgctggcatccc

SEQ ID NO: 62 - RNA, 23 nt, synthetic construct
  source 1..23: mol_type = other RNA
tactcagagaggcactggcatgt

SEQ ID NO: 63 - RNA, 20 nt, synthetic construct
  source 1..20: mol_type = other RNA
acggtcctctgcccactcag

SEQ ID NO: 64 - RNA, 22 nt, synthetic construct
  source 1..22: mol_type = other RNA
tgggagacagcagagagaggct

SEQ ID NO: 65 - RNA, 22 nt, synthetic construct
  source 1..22: mol_type = other RNA
aggggtgagccccacagagtct

SEQ ID NO: 66 - RNA, 22 nt, synthetic construct
  source 1..22: mol_type = other RNA
cttgcctttgcacttgggggtt

SEQ ID NO: 67 - RNA, 22 nt, synthetic construct
  source 1..22: mol_type = other RNA
ttgaccgaggatctggccccag

SEQ ID NO: 68 - RNA, 23 nt, synthetic construct
  source 1..23: mol_type = other RNA
ttctagcagtgccggcaccccat

SEQ ID NO: 69 - RNA, 21 nt, synthetic construct
  source 1..21: mol_type = other RNA
gcggagcaggtcttggatcat

SEQ ID NO: 70 - RNA, 21 nt, synthetic construct
  source 1..21: mol_type = other RNA
gatgggagtcagtggccgtgg

SEQ ID NO: 71 - RNA, 18 nt, synthetic construct
  source 1..18: mol_type = other RNA
tgagagtgggttcaagtc

SEQ ID NO: 72 - RNA, 21 nt, synthetic construct
  source 1..21: mol_type = other RNA
caaggctgggagatgctctga

SEQ ID NO: 73 - RNA, 23 nt, synthetic construct
  source 1..23: mol_type = other RNA
cacctgcagtgccggcatcccat

SEQ ID NO: 74 - RNA, 22 nt, synthetic construct
  source 1..22: mol_type = other RNA
tgcatcacattgccagtcctga

SEQ ID NO: 75 - RNA, 24 nt, synthetic construct
  source 1..24: mol_type = other RNA
cttggctgtggctgccccaggttc

SEQ ID NO: 76 - RNA, 25 nt, synthetic construct
  source 1..25: mol_type = other RNA
accacacggggcaccgccgggctgg

SEQ ID NO: 77 - RNA, 22 nt, synthetic construct
  source 1..22: mol_type = other RNA
tggggctgcctgccatgtgtgg

SEQ ID NO: 78 - RNA, 22 nt, synthetic construct
  source 1..22: mol_type = other RNA
ttgagggcatcttaagagagga

SEQ ID NO: 79 - RNA, 22 nt, synthetic construct
  source 1..22: mol_type = other RNA
caaaagttcatggaagtggaag

SEQ ID NO: 80 - RNA, 21 nt, synthetic construct
  source 1..21: mol_type = other RNA
gggctgtatgtgtgcttttgc

SEQ ID NO: 81 - RNA, 21 nt, synthetic construct
  source 1..21: mol_type = other RNA
ggggctggcgctgtggtgcag

SEQ ID NO: 82 - RNA, 22 nt, synthetic construct
  source 1..22: mol_type = other RNA
agggccggcactgtagtatagt

SEQ ID NO: 83 - RNA, 21 nt, synthetic construct
  source 1..21: mol_type = other RNA
agacaggaagggtgtgtctgt

SEQ ID NO: 84 - RNA, 21 nt, synthetic construct
  source 1..21: mol_type = other RNA
caatcagcctactgggaacat

SEQ ID NO: 85 - RNA, 21 nt, synthetic construct
  source 1..21: mol_type = other RNA
tctgtctgccctccgcttgct

SEQ ID NO: 86 - RNA, 25 nt, synthetic construct
  source 1..25: mol_type = other RNA
acctgcatgttagtctaatgttctg

SEQ ID NO: 87 - RNA, 22 nt, synthetic construct
  source 1..22: mol_type = other RNA
cccacccacccctgtgagatcg

SEQ ID NO: 88 - RNA, 21 nt, synthetic construct
  source 1..21: mol_type = other RNA
tgacccccggccggaccccac

SEQ ID NO: 89 - RNA, 21 nt, synthetic construct
  source 1..21: mol_type = other RNA
aagggatcggcattctggcat

SEQ ID NO: 90 - RNA, 21 nt, synthetic construct
  source 1..21: mol_type = other RNA
agtccagggcttagtcagcgg

SEQ ID NO: 91 - RNA, 21 nt, synthetic construct
  source 1..21: mol_type = other RNA
tcgtatccacctctgttgcag

SEQ ID NO: 92 - RNA, 22 nt, synthetic construct
  source 1..22: mol_type = other RNA
atcatgtatgatactgcacaca

SEQ ID NO: 93 - RNA, 22 nt, synthetic construct
  source 1..22: mol_type = other RNA
tattcaagcccagcggaggtac

SEQ ID NO: 94 - RNA, 22 nt, synthetic construct
  source 1..22: mol_type = other RNA
tatgtttgctgttttgtgagac

SEQ ID NO: 95 - RNA, 24 nt, synthetic construct
  source 1..24: mol_type = other RNA
cccccaaggacactgcctgcctac

SEQ ID NO: 96 - RNA, 23 nt, synthetic construct
  source 1..23: mol_type = other RNA
tagggagagaatgagtggactga

SEQ ID NO: 97 - RNA, 22 nt, synthetic construct
  source 1..22: mol_type = other RNA
gtcgcacgcacgcccgcctcgg

SEQ ID NO: 98 - RNA, 18 nt, synthetic construct
  source 1..18: mol_type = other RNA
ggtgctgtaggctttaaa

SEQ ID NO: 99 - RNA, 20 nt, synthetic construct
  source 1..20: mol_type = other RNA
gaagtcgcagctctctgggc

SEQ ID NO: 100 - RNA, 18 nt, synthetic construct
  source 1..18: mol_type = other RNA
gtctgagcggcggccact

SEQ ID NO: 101 - RNA, 22 nt, synthetic construct
  source 1..22: mol_type = other RNA
tgtctcctgtgcacttcccagg

SEQ ID NO: 102 - RNA, 22 nt, synthetic construct
  source 1..22: mol_type = other RNA
tcaaaaagtcggcgggaaatgc

SEQ ID NO: 103 - RNA, 22 nt, synthetic construct
  source 1..22: mol_type = other RNA
ccgtgaccgagagcgagaacga

SEQ ID NO: 104 - RNA, 21 nt, synthetic construct
  source 1..21: mol_type = other RNA
ttcctgtgctgttctcggtag

SEQ ID NO: 105 - RNA, 21 nt, synthetic construct
  source 1..21: mol_type = other RNA
ttcctgtgctgttctcggtag

SEQ ID NO: 106 - RNA, 21 nt, synthetic construct
  source 1..21: mol_type = other RNA
tcaaaggaagaagactggcgt

SEQ ID NO: 107 - RNA, 21 nt, synthetic construct
  source 1..21: mol_type = other RNA
ttggctgagacagtgtgacat

SEQ ID NO: 108 - RNA, 21 nt, synthetic construct
  source 1..21: mol_type = other RNA
caatcagcctactgggaacat

SEQ ID NO: 109 - RNA, 22 nt, synthetic construct
  source 1..22: mol_type = other RNA
aggggtgagccccacagagtct

SEQ ID NO: 110 - RNA, 20 nt, synthetic construct
  source 1..20: mol_type = other RNA
tgggcccacagagactctgg

SEQ ID NO: 111 - RNA, 21 nt, synthetic construct
  source 1..21: mol_type = other RNA
ttcctgtgctgctctgagtag

SEQ ID NO: 112 - RNA, 22 nt, synthetic construct
  source 1..22: mol_type = other RNA
cctagagtgtcggcatcccttt

SEQ ID NO: 113 - RNA, 21 nt, synthetic construct
  source 1..21: mol_type = other RNA
ttctgcctgtgtaaggaatgc

SEQ ID NO: 114 - RNA, 22 nt, synthetic construct
  source 1..22: mol_type = other RNA
atcatgtatgatactgcacaca

SEQ ID NO: 115 - RNA, 22 nt, synthetic construct
  source 1..22: mol_type = other RNA
tgtggggaccattgttgagtgc

SEQ ID NO: 116 - RNA, 23 nt, synthetic construct
  source 1..23: mol_type = other RNA
cgcaggtctggggtctgagctgc

SEQ ID NO: 117 - RNA, 21 nt, synthetic construct
  source 1..21: mol_type = other RNA
atgattttcagctaagaatgg

SEQ ID NO: 118 - RNA, 18 nt, synthetic construct
  source 1..18: mol_type = other RNA
gtgtatgatgactggatt

SEQ ID NO: 119 - RNA, 21 nt, synthetic construct
  source 1..21: mol_type = other RNA
gcggagcaggtcttggatcat

SEQ ID NO: 120 - RNA, 23 nt, synthetic construct
  source 1..23: mol_type = other RNA
tactcagagcgggactggaatgt

SEQ ID NO: 121 - RNA, 23 nt, synthetic construct
  source 1..23: mol_type = other RNA
tactcagagcgggactggaatgt

SEQ ID NO: 122 - RNA, 22 nt, synthetic construct
  source 1..22: mol_type = other RNA
catccagaagtccagggacttt

SEQ ID NO: 123 - RNA, 21 nt, synthetic construct
  source 1..21: mol_type = other RNA
ttcctggaacgctcggggagc

SEQ ID NO: 124 - RNA, 23 nt, synthetic construct
  source 1..23: mol_type = other RNA
tacctagaacagcaacggagtgt

SEQ ID NO: 125 - RNA, 22 nt, synthetic construct
  source 1..22: mol_type = other RNA
tgaacctgtgatgccagcatcc

SEQ ID NO: 126 - RNA, 22 nt, synthetic construct
  source 1..22: mol_type = other RNA
tatgtttgctgttttgtgagac

SEQ ID NO: 127 - RNA, 22 nt, synthetic construct
  source 1..22: mol_type = other RNA
cttcctggctgacgtggagtcc

SEQ ID NO: 128 - RNA, 20 nt, synthetic construct
  source 1..20: mol_type = other RNA
ggcagggcctgcttccttcc

SEQ ID NO: 129 - RNA, 20 nt, synthetic construct
  source 1..20: mol_type = other RNA
cgtctgggctggcccgagcg

SEQ ID NO: 130 - RNA, 23 nt, synthetic construct
  source 1..23: mol_type = other RNA
tacctagaacagcaatggagtgt

SEQ ID NO: 131 - RNA, 22 nt, synthetic construct
  source 1..22: mol_type = other RNA
cctctgcactctctctcaccag

SEQ ID NO: 132 - RNA, 21 nt, synthetic construct
  source 1..21: mol_type = other RNA
tctggcttcaaggtgctgtgt

SEQ ID NO: 133 - RNA, 18 nt, synthetic construct
  source 1..18: mol_type = other RNA
acacagcagactcagagg